SEQUENCE LISTING (77 sequences)

SEQ ID NO: 1 - protein, 109 aa, Homo sapiens
DFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 2 - protein, 6 aa, Artificial Sequence
  Description of Artificial Sequence Synthetic peptide
KXXPXN

SEQ ID NO: 3 - protein, 17 aa, Homo sapiens
PCCTPTKMSPINMLYFN

SEQ ID NO: 4 - DNA, 747 bp, Homo sapiens
caggtgcagctggtgcaatctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagagacgagaactgggggttcgacccctggggccagggaaccctggtcaccgtctcgagtggaggcggcggttcaggcggaggtggctctggcggtggcggaagtgcactttcctatgagctgactcagccaccctcagtgtccgtgtctccaggacagacagccaccattacctgctctggacatgcactgggggacaaatttgtttcctggtatcagcagggatcaggccagtcccctgtattggtcatctatgacgatacccagcggccctcagggatccctgggcgattctctggctccaactctgggaacacagccactctgaccatcagcgggacccaggctatggatgaggctgactatttttgtcaggcgtgggacagcagcttcgtattcggcggagggaccaaggtcaccgtcctaggtgcggccgcacatcatcatcaccatcac

SEQ ID NO: 5 - DNA, 774 bp, Homo sapiens
caggtcaccttgaaggagtctgggggaggcttggtacagcctggggggtccctgagactctcctgtgcagcctctggattcacctttagtagatatgtcatcaactgggtccgccaggctccagggaaggggctggaatgggtctcagctattagtgttactggtggtagcacggcctacgcagactccgtgaggggccggttcaccatctccagagacaattccaagaacacgctgtatttgcaaatgaatagcctgagagccgaggacacggccgtatattactgtacgaaaggacagtgggaacggggaagttactactttgactactggggccggggaaccctggtcaccgtctcgagtggaggcggcggttcaggcggaggtggctctggcggtggcggaagtgcacagtctgtgctgacgcagccgccctcagtgtctggggccccagggcagagggtcaccatctcctgcactgggagcagctccaacatcggggacggttatgatgtacactggtatcagcagcttccaggaacagcccccaaactcctcatctatggtaacagtcatcggccctcaggggtccctgaccgattctctggctccaagtctgacacctctgcctccctggccatcactgggctccaggttgaggatgaggctgattatttctgccactcctatgacggcagtgtgagtggctggattttcggcggagggaccaagctgaccgtcctaggtgcggccgcacatcatcatcaccatcac

SEQ ID NO: 6 - DNA, 786 bp, Homo sapiens
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgagagaatggggccctgtactggtggaagctgctacgacacccttggcaactggggccggggcaccctggtcaccgtctcgagtggaggcggcggttcaggcggaggtggctctggcggtggcggaagtgcacagtctgtgctgacgcagccgccctcagtgtctggggccccagggcagagggtcaccatctcctgcactgggagcagctccaacatcggggcaggttatgatgtacactggtaccagcaacttccaggcgcggcccccaaactcctcatcaggggtaatggcaatcggccctcaggggtccctgaccgattctctgtctccaagtctggctactcagcctccctggccatcactgggctgcagcctgccgatgagggtgtttattactgccagtcctatgacagcagtctgagtggttcgaaggtgttcggccaagggaccaagctgaccgtcctaggtgcggccgcacatcatcatcaccatcac

SEQ ID NO: 7 - protein, 249 aa, Homo sapiens
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDENWGFDPWGQGTLVTVSSGGGGSGGGGSGGGGSALSYELTQPPSVSVSPGQTATITCSGHALGDKFVSWYQQGSGQSPVLVIYDDTQRPSGIPGRFSGSNSGNTATLTISGTQAMDEADYFCQAWDSSFVFGGGTKVTVLGAAAHHHHHH

SEQ ID NO: 8 - protein, 258 aa, Homo sapiens
QVTLKESGGGLVQPGGSLRLSCAASGFTFSRYVINWVRQAPGKGLEWVSAISVTGGSTAYADSVRGRFTISRDNSKNTLYLQMNSLRAEDTAVYYCTKGQWERGSYYFDYWGRGTLVTVSSGGGGSGGGGSGGGGSAQSVLTQPPSVSGAPGQRVTISCTGSSSNIGDGYDVHWYQQLPGTAPKLLIYGNSHRPSGVPDRFSGSKSDTSASLAITGLQVEDEADYFCHSYDGSVSGWIFGGGTKLTVLGAAAHHHHHH

SEQ ID NO: 9 - protein, 262 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCERMGPCTGGSCYDTLGNWGRGTLVTVSSGGGGSGGGGSGGGGSAQSVLTQPPSVSGAPGQRVTISCTGSSSNIGAGYDVHWYQQLPGAAPKLLIRGNGNRPSGVPDRFSVSKSGYSASLAITGLQPADEGVYYCQSYDSSLSGSKVFGQGTKLTVLGAAAHHHHHH

SEQ ID NO: 10 - DNA, 351 bp, Homo sapiens
caggtgcagctggtgcaatctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagagacgagaactgggggttcgacccctggggccagggaaccctggtcaccgtctcgagt

SEQ ID NO: 11 - DNA, 363 bp, Homo sapiens
caggtcaccttgaaggagtctgggggaggcttggtacagcctggggggtccctgagactctcctgtgcagcctctggattcacctttagtagatatgtcatcaactgggtccgccaggctccagggaaggggctggaatgggtctcagctattagtgttactggtggtagcacggcctacgcagactccgtgaggggccggttcaccatctccagagacaattccaagaacacgctgtatttgcaaatgaatagcctgagagccgaggacacggccgtatattactgtacgaaaggacagtgggaacggggaagttactactttgactactggggccggggaaccctggtcaccgtctcgagt

SEQ ID NO: 12 - DNA, 372 bp, Homo sapiens
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgagagaatggggccctgtactggtggaagctgctacgacacccttggcaactggggccggggcaccctggtcaccgtctcgagt

SEQ ID NO: 13 - protein, 117 aa, Homo sapiens
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDENWGFDPWGQGTLVTVSS

SEQ ID NO: 14 - protein, 121 aa, Homo sapiens
QVTLKESGGGLVQPGGSLRLSCAASGFTFSRYVINWVRQAPGKGLEWVSAISVTGGSTAYADSVRGRFTISRDNSKNTLYLQMNSLRAEDTAVYYCTKGQWERGSYYFDYWGRGTLVTVSS

SEQ ID NO: 15 - protein, 124 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCERMGPCTGGSCYDTLGNWGRGTLVTVSS

SEQ ID NO: 16 - DNA, 315 bp, Homo sapiens
tcctatgagctgactcagccaccctcagtgtccgtgtctccaggacagacagccaccattacctgctctggacatgcactgggggacaaatttgtttcctggtatcagcagggatcaggccagtcccctgtattggtcatctatgacgatacccagcggccctcagggatccctgggcgattctctggctccaactctgggaacacagccactctgaccatcagcgggacccaggctatggatgaggctgactatttttgtcaggcgtgggacagcagcttcgtattcggcggagggaccaaggtcaccgtccta

SEQ ID NO: 17 - DNA, 336 bp, Homo sapiens
cagtctgtgctgacgcagccgccctcagtgtctggggccccagggcagagggtcaccatctcctgcactgggagcagctccaacatcggggacggttatgatgtacactggtatcagcagcttccaggaacagcccccaaactcctcatctatggtaacagtcatcggccctcaggggtccctgaccgattctctggctccaagtctgacacctctgcctccctggccatcactgggctccaggttgaggatgaggctgattatttctgccactcctatgacggcagtgtgagtggctggattttcggcggagggaccaagctgaccgtcctaggt

SEQ ID NO: 18 - DNA, 336 bp, Homo sapiens
cagtctgtgctgacgcagccgccctcagtgtctggggccccagggcagagggtcaccatctcctgcactgggagcagctccaacatcggggcaggttatgatgtacactggtaccagcaacttccaggcgcggcccccaaactcctcatcaggggtaatggcaatcggccctcaggggtccctgaccgattctctgtctccaagtctggctactcagcctccctggccatcactgggctgcagcctgccgatgagggtgtttattactgccagtcctatgacagcagtctgagtggttcgaaggtgttcggccaagggaccaagctgaccgtccta

SEQ ID NO: 19 - protein, 105 aa, Homo sapiens
SYELTQPPSVSVSPGQTATITCSGHALGDKFVSWYQQGSGQSPVLVIYDDTQRPSGIPGRFSGSNSGNTATLTISGTQAMDEADYFCQAWDSSFVFGGGTKVTVL

SEQ ID NO: 20 - protein, 111 aa, Homo sapiens
QSVLTQPPSVSGAPGQRVTISCTGSSSNIGDGYDVHWYQQLPGTAPKLLIYGNSHRPSGVPDRFSGSKSDTSASLAITGLQVEDEADYFCHSYDGSVSGWIFGGGTKLTVL

SEQ ID NO: 21 - protein, 112 aa, Homo sapiens
QSVLTQPPSVSGAPGQRVTISCTGSSSNIGAGYDVHWYQQLPGAAPKLLIRGNGNRPSGVPDRFSVSKSGYSASLAITGLQPADEGVYYCQSYDSSLSGSKVFGQGTKLTVL

SEQ ID NO: 22 - DNA, 747 bp, Homo sapiens
caggtgcagctggtgcaatctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagagacgagaactgggggttcgacccctggggccagggaaccctggtcaccgtctcgagtggaggcggcggttcaggcggaggtggctctggcggtggcggaagtgcactttcctatgagctgactcagccaccctcagtgtccgtgtctccaggacagacagccagcattacctgctctggacatgcactgggggacaaatttgtttcctggtatcagcagaagccaggccagtcccctgtattggtcatctatgacgatacccagcggccctcagggatccctgagcgattctctggctccaactctgggaacacagccactctgaccatcagcgggacccaggctatggatgaggctgactattactgtcaggcgtgggacagcagcttcgtattcggcggagggaccaaggtcaccgtcctaggtgcggccgcacatcaccatcaccatcac

SEQ ID NO: 23 - DNA, 774 bp, Homo sapiens
gaggtccagttgttggagtctgggggaggcttggtacagcctggggggtccctgagactctcctgtgcagcctctggattcacctttagtagatatgtcatcaactgggtccgccaggctccagggaaggggctggaatgggtctcagctattagtgttactggtggtagcacggcctacgcagactccgtgaggggccggttcaccatctccagagacaattccaagaacacgctgtatttgcaaatgaatagcctgagagccgaggacacggccgtatattactgtgcgaaaggacagtgggaacggggaagttactactttgactactggggccggggaaccctggtcaccgtctcgagtggaggcggcggttcaggcggaggtggctctggcggtggcggaagtgcacagtctgtgctgacgcagccgccctcagtgtctggggccccagggcagagggtcaccatctcctgcactgggagcagctccaacatcggggacggttatgatgtacactggtatcagcagcttccaggaacagcccccaaactcctcatctatggtaacagtcatcggccctcaggggtccctgaccgattctctggctccaagtctggtacctctgcctccctggccatcactgggctccaggctgaggatgaggctgattattactgccactcctatgacggcagtgtgagtggctggattttcggcggagggaccaagctgaccgtcctaggtgcggccgcacatcatcatcaccatcac

SEQ ID NO: 24 - protein, 249 aa, Homo sapiens
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDENWGFDPWGQGTLVTVSSGGGGSGGGGSGGGGSALSYELTQPPSVSVSPGQTASITCSGHALGDKFVSWYQQKPGQSPVLVIYDDTQRPSGIPERFSGSNSGNTATLTISGTQAMDEADYYCQAWDSSFVFGGGTKVTVLGAAAHHHHHH

SEQ ID NO: 25 - protein, 258 aa, Homo sapiens
EVQLLESGGGLVQPGGSLRLSCAASGFTFSRYVINWVRQAPGKGLEWVSAISVTGGSTAYADSVRGRFTISRDNSKNTLYLQMNSLRAEDTAVYYCAKGQWERGSYYFDYWGRGTLVTVSSGGGGSGGGGSGGGGSAQSVLTQPPSVSGAPGQRVTISCTGSSSNIGDGYDVHWYQQLPGTAPKLLIYGNSHRPSGVPDRFSGSKSGTSASLAITGLQAEDEADYYCHSYDGSVSGWIFGGGTKLTVLGAAAHHHHHH

SEQ ID NO: 26 - DNA, 351 bp, Homo sapiens; identical to SEQ ID NO: 10

SEQ ID NO: 27 - DNA, 363 bp, Homo sapiens
gaggtccagttgttggagtctgggggaggcttggtacagcctggggggtccctgagactctcctgtgcagcctctggattcacctttagtagatatgtcatcaactgggtccgccaggctccagggaaggggctggaatgggtctcagctattagtgttactggtggtagcacggcctacgcagactccgtgaggggccggttcaccatctccagagacaattccaagaacacgctgtatttgcaaatgaatagcctgagagccgaggacacggccgtatattactgtgcgaaaggacagtgggaacggggaagttactactttgactactggggccggggaaccctggtcaccgtctcgagt

SEQ ID NO: 28 - protein, 117 aa, Homo sapiens; identical to SEQ ID NO: 13

SEQ ID NO: 29 - protein, 121 aa, Homo sapiens
EVQLLESGGGLVQPGGSLRLSCAASGFTFSRYVINWVRQAPGKGLEWVSAISVTGGSTAYADSVRGRFTISRDNSKNTLYLQMNSLRAEDTAVYYCAKGQWERGSYYFDYWGRGTLVTVSS

SEQ ID NO: 30 - DNA, 315 bp, Homo sapiens
tcctatgagctgactcagccaccctcagtgtccgtgtctccaggacagacagccagcattacctgctctggacatgcactgggggacaaatttgtttcctggtatcagcagaagccaggccagtcccctgtattggtcatctatgacgatacccagcggccctcagggatccctgagcgattctctggctccaactctgggaacacagccactctgaccatcagcgggacccaggctatggatgaggctgactattactgtcaggcgtgggacagcagcttcgtattcggcggagggaccaaggtcaccgtccta

SEQ ID NO: 31 - DNA, 333 bp, Homo sapiens
cagtctgtgctgacgcagccgccctcagtgtctggggccccagggcagagggtcaccatctcctgcactgggagcagctccaacatcggggacggttatgatgtacactggtatcagcagcttccaggaacagcccccaaactcctcatctatggtaacagtcatcggccctcaggggtccctgaccgattctctggctccaagtctggtacctctgcctccctggccatcactgggctccaggctgaggatgaggctgattattactgccactcctatgacggcagtgtgagtggctggattttcggcggagggaccaagctgaccgtccta

SEQ ID NO: 32 - protein, 105 aa, Homo sapiens
SYELTQPPSVSVSPGQTASITCSGHALGDKFVSWYQQKPGQSPVLVIYDDTQRPSGIPERFSGSNSGNTATLTISGTQAMDEADYYCQAWDSSFVFGGGTKVTVL

SEQ ID NO: 33 - protein, 111 aa, Homo sapiens
QSVLTQPPSVSGAPGQRVTISCTGSSSNIGDGYDVHWYQQLPGTAPKLLIYGNSHRPSGVPDRFSGSKSGTSASLAITGLQAEDEADYYCHSYDGSVSGWIFGGGTKLTVL

SEQ ID NO: 34 - protein, 5 aa, Homo sapiens
SYYMH

SEQ ID NO: 35 - protein, 5 aa, Homo sapiens
RYVIN

SEQ ID NO: 36 - protein, 5 aa, Homo sapiens
SYAMS

SEQ ID NO: 37 - protein, 17 aa, Homo sapiens
IINPSGGSTSYAQKFQG

SEQ ID NO: 38 - protein, 17 aa, Homo sapiens
AISVTGGSTAYADSVRG

SEQ ID NO: 39 - protein, 17 aa, Homo sapiens
AISGSGGSTYYADSVKG

SEQ ID NO: 40 - protein, 8 aa, Homo sapiens
DENWGFDP

SEQ ID NO: 41 - protein, 12 aa, Homo sapiens
GQWERGSYYFDY

SEQ ID NO: 42 - protein, 15 aa, Homo sapiens
MGPCTGGSCYDTLGN

SEQ ID NO: 43 - protein, 11 aa, Homo sapiens
SGHALGDKFVS

SEQ ID NO: 44 - protein, 14 aa, Homo sapiens
TGSSSNIGDGYDVH

SEQ ID NO: 45 - protein, 14 aa, Homo sapiens
TGSSSNIGAGYDVH

SEQ ID NO: 46 - protein, 7 aa, Homo sapiens
DDTQRPS

SEQ ID NO: 47 - protein, 7 aa, Homo sapiens
GNSHRPS

SEQ ID NO: 48 - protein, 7 aa, Homo sapiens
GNGNRPS

SEQ ID NO: 49 - protein, 7 aa, Homo sapiens
QAWDSSF

SEQ ID NO: 50 - protein, 6 aa, Homo sapiens
HSYDGS

SEQ ID NO: 51 - protein, 12 aa, Homo sapiens
QSYDSSLSGSKV

SEQ ID NO: 52 - DNA, 320 bp, Homo sapiens
gtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatag

SEQ ID NO: 53 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 54 - DNA, 992 bp, Homo sapiens
cctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 55 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 5 aa, Homo sapiens
  MOD_RES (4): Variable amino acid
DEHXT

SEQ ID NO: 57 - protein, 5 aa, Homo sapiens
DEHST

SEQ ID NO: 58 - protein, 15 aa, Homo sapiens
  MOD_RES (10): Variable amino acid
DFGLDCDEHXTESRC

SEQ ID NO: 59 - protein, 15 aa, Homo sapiens
DFGLDCDEHSTESRC

SEQ ID NO: 60 - protein, 378 aa, Artificial Sequence
  Description of Artificial Sequence Synthetic chimeric fusion protein
MKFLVNVALVFMVVYISYIYATSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGRGDDDDKTRSRDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPPK

SEQ ID NO: 61 - DNA, 1134 bp, Artificial Sequence
  Description of Artificial Sequence Synthetic chimeric fusion nucleotide sequence
atgaaattcttagtcaacgttgcccttgtttttatggtcgtgtacatttcttacatctatgcgactagtgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccggcggccgcggagacgacgacgacaagacgcgttctagagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagtccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgcctaaa

SEQ ID NO: 62 - protein, 512 aa, Homo sapiens
MTAPWVALALLWGSLCAGSGRGEAETRECIYYNANWELERTNQSGLERCEGEQDKRLHCYASWANSSGTIELVKKGCWLDDFNCYDRQECVATEENPQVYFCCCEGNFCNERFTHLPEAGGPEVTYEPPPTAPTLLTVLAYSLLPIGGLSLIVLLAFWMYRHRKPPYGHVDIHEDPGPPPPSPLVGLKPLQLLEIKARGRFGCVWKAQLMNDFVAVKIFPLQDKQSWQSEREIFSTPGMKHENLLQFIAAEKRGSNLEVELWLITAFHDKGSLTDYLKGNIITWNELCHVAETMSRGLSYLHEDVPWCRGEGHKPSIAHRDFKSKNVLLKSDLTAVLADFGLAVRFEPGKPPGDTHGQVGTRRYMAPEVLEGAINFQRDAFLRIDMYAMGLVLWELVSRCKAADGPVDEYMLPFEEEIGQHPSLEELQEVVVHKKMRPTIKDHWLKHPGLAQLCVTIEECWDHDAEARLSAGCVEERVSLIRRSVNGTTSDCLVSLVTSVTNVDLPPKESSI

SEQ ID NO: 63 - protein, 4 aa, Artificial Sequence
  Description of Artificial Sequence Synthetic gly-ser linker
GSGS

SEQ ID NO: 64 - protein, 4 aa, Artificial Sequence
  Description of Artificial Sequence Synthetic Enterokinase cleavage site linking sequence
DDDK

SEQ ID NO: 65 - protein, 243 aa, Homo sapiens
NENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRR

SEQ ID NO: 66 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 227 aa, Homo sapiens
DKTHTCPPCPAPEALGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 50 aa, Artificial Sequence
  Description of Artificial Sequence Synthetic mutant peptide portion of human myostatin
KDVIRQLLPKAPPLRELIDQYDVQRADSSDGSLEDDDYHATTETIITMPT

SEQ ID NO: 69 - protein, 40 aa, Artificial Sequence
  Description of Artificial Sequence Synthetic mutant peptide portion of human myostatin
QLLPKAPPLRELIDQYDVQRADSSDGSLEDDDYHATTETI

SEQ ID NO: 70 - protein, 30 aa, Artificial Sequence
  Description of Artificial Sequence Synthetic mutant peptide portion of human myostatin
APPLRELIDQYDVQRADSSDGSLEDDDYHA

SEQ ID NO: 71 - protein, 20 aa, Artificial Sequence
  Description of Artificial Sequence Synthetic mutant peptide portion of human myostatin
ELIDQYDVQRADSSDGSLED

SEQ ID NO: 72 - protein, 10 aa, Artificial Sequence
  Description of Artificial Sequence Synthetic mutant peptide portion of human myostatin
YDVQRADSSD

SEQ ID NO: 73 - protein, 50 aa, Homo sapiens
KDVIRQLLPKAPPLRELIDQYDVQRDDSSDGSLEDDDYHATTETIITMPT

SEQ ID NO: 74 - protein, 40 aa, Homo sapiens
QLLPKAPPLRELIDQYDVQRDDSSDGSLEDDDYHATTETI

SEQ ID NO: 75 - protein, 30 aa, Homo sapiens
APPLRELIDQYDVQRDDSSDGSLEDDDYHA

SEQ ID NO: 76 - protein, 20 aa, Homo sapiens
ELIDQYDVQRDDSSDGSLED

SEQ ID NO: 77 - protein, 10 aa, Homo sapiens
YDVQRDDSSD

(19) United States
(12) Patent Application Publication
(10) Pub. No.: US 2006/0034831 A1
(43) Pub. Date: Feb. 16, 2006

(54) Combination therapy for diabetes, obesity and cardiovascular diseases using GDF-8 inhibitors

(75) Inventor: James F. Tobin, Newton, MA (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Wyeth

(21) Appl. No.: 11/201,825

(22) Filed: Aug. 11, 2005

Related U.S. Application Data
(60) Provisional application No. 60/600,784, filed on Aug. 12, 2004.

(51) Int. Cl. A61K 39/395; A61K 38/28; A61K 31/426; A61K 31/401; A61K 31/175; A61K 31/155

(52) U.S. Cl. 424/130.1; 514/3; 514/369; 514/423; 514/635; 514/592

(57) ABSTRACT

A method of treating obesity, cardiovascular diseases, and disorders of insulin metabolism in a subject, comprising administering to the subject a therapeutically effective amount of a GDF-8 inhibitor, and a therapeutically effective amount of at least one other therapeutic agent which treats the targeted syndrome.

### RELATED CASES

This application claims the benefit of U.S. Provisional Application No. 60/600,784, filed Aug. 12, 2004, the contents of which are incorporated herein in their entirety by reference.

### TECHNICAL FIELD

This invention relates to methods of treating at least one of obesity, cardiovascular diseases, and disorders of insulin metabolism, such as diabetes and syndrome X, using combination therapy. The novel combination therapy employs at least one inhibitor of growth and differentiation factor-8 (GDF-8) and at least one other therapeutic agent.

### BACKGROUND OF THE INVENTION

Growth and differentiation factor-8 (GDF-8), also known as myostatin, is a secreted protein and is a member of the transforming growth factor-beta (TGF-β) superfamily of structurally related growth factors, all of which possess physiologically important growth-regulatory and morphogenetic properties (Kingsley et al., Genes Dev. 8:133-146 (1994); Hoodless et al., Curr. Topics Microbiol. Immunol. 228:235-272 (1998)). Similarly to TGF-β, human GDF-8 is synthesized as a 375 amino acid long precursor protein. The precursor GDF-8 protein forms a homodimer. During processing the amino-terminal propeptide is cleaved off at Arg-266. The cleaved propeptide, known as the “latency-associated peptide” (LAP), may remain noncovalently bound to the homodimer, thereby inactivating the complex (Miyazono et al., J. Biol. Chem. 263:6407-6415 (1988); Wakefield et al., J. Biol. Chem. 263:7646-7654 (1988); Brown et al., Growth Factors 3:35-43 (1990); and Thies et al., Growth Factors 18:251-259 (2001)). The complex of mature GDF-8 with propeptide is commonly referred to as the “small latent complex” (Gentry et al., Biochemistry 29:6851-6857 (1990); Derynck et al., Nature, 316:701-705 (1995); and Massague, Ann. Rev. Cell Biol. 12:597-641 (1990)). Other proteins are also known to bind to mature GDF-8 and inhibit its biological activity. Such inhibitory proteins include follistatin and follistatin-related proteins (Gamer et al., Dev. Biol., 208:222-232 (1999)).

An alignment of deduced amino acid sequences from various species demonstrates that GDF-8 is highly conserved throughout evolution (McPherron et al., Proc. Nat. Acad. Sci. U.S.A. 94:12457-12461 (1997)). In fact, the sequences of human, mouse, rat, porcine, and chicken GDF-8 are 100% identical in the C-terminal region, while those of baboon, bovine, and ovine differ by 3 amino acids or less. The zebrafish GDF-8 is the most diverged; however, it is still 88% identical to human.

The high degree of conservation suggests that GDF-8 has an essential function. GDF-8 is highly expressed in the developing and adult skeletal muscle and was found to be involved in the regulation of critical biological processes in the muscle and in osteogenesis. For example, GDF-8 knockout transgenic mice are characterized by a marked hypertrophy and hyperplasia of the skeletal muscle (McPherron et al., Nature 387:83-90 (1997)) and altered cortical bone structure (Hamrick et al., Bone 27:343-349 (2000)). Similarly, increases in skeletal muscle mass are evident in naturally occurring mutations of GDF-8 in cattle (Ashmore et al., Growth, 38:501-507 (1974); Swatland et al., J. Anim. Sci. 38:752-757 (1994); McPherron et al., Proc. Nat. Acad. Sci. U.S.A. 94:12457-12461 (1997); and Kambadur et al., Genome Res. 7:910-915 (1997)). Studies have indicated that muscle wasting associated with HIV-infection is accompanied by an increase in GDF-8 expression (Gonzalez-Cadavid et al., Proc. Nat. Acad. Sci. U.S.A. 95:14938-14943 (1998)). GDF-8 has also been implicated in the production of muscle-specific enzymes (e.g., creatine kinase) and proliferation of myoblast cells (WO 00/43781). In addition to its growth-regulatory and morphogenetic properties, GDF-8 is thought to be also involved in a number of other physiological processes, including glucose homeostasis in the development of type 2 diabetes, impaired glucose tolerance, metabolic syndromes (e.g., syndrome X), insulin resistance induced by trauma, such as burns or nitrogen imbalance, and adipose tissue disorders (e.g., obesity) (Kim et al. BBRC 281:902-906 (2001)).

Other studies extend the role of GDF-8 in adipogenesis and glucose homeostasis. For example, injection of GDF-8 secreting tumor cells into mice increases their level of blood sugar (hyperglycemia) and decreases their weight and muscle mass. Also GDF-8 blocks insulin-induced expression of GLUT4, and it blocks insulin-mediated differentiation of pre-adipocytes. Collectively, the GDF-8 studies suggest that inhibition of GDF-8 would reduce blood sugar and body fat, and increase insulin-mediated transport of glucose, conditions that may benefit a patient having or who may ultimately acquire type 2 diabetes or syndrome X, or other syndromes involving glucose homeostasis.

Obesity, cardiovascular diseases, and/or disorders of insulin metabolism, such as diabetes and/or syndrome X have been treated using a number of different therapies. These therapies include angiotensin converting enzyme inhibitors, sulfonylurea agents, antilipemic agents, biguanide agents, thiazolidinedione agents, insulin, alpha-glucosidase inhibitors, and aldose reductase inhibitors, although not all the therapies have been recognized for the treatment of all the diseases and disorders described. These therapies work through a variety of mechanisms, none of which are related to GDF-8.

### SUMMARY OF THE INVENTION

The present invention relates to methods of treating at least one of obesity, cardiovascular diseases, and disorders of insulin metabolism, including diabetes and syndrome X, by administering an effective amount a GDF-8 inhibitor in combination with at least one other therapeutic agent.

At least one of obesity, cardiovascular diseases, and disorders of insulin metabolism, such as diabetes and syndrome X, may be treated with inhibitors of GDF-8 in combination with other therapeutic agents that treat these targeted syndromes. This approach to treatment is called combination therapy. A variety of other therapeutics have been used to treat the different causes and diseases associated these targeted syndromes, including agents to stimulate glucose transport (e.g., insulin, sulfonylurea agents, biguanide agents, thiazolidinedione agents), agents to control blood sugar (e.g., alpha-glucosidase inhibitors), agents to improve cardiovascular health (e.g., antilipemic agents and ACE inhibitors), and agents to reduce toxic sorbitol production in the eye and nerves (e.g., aldose reductase inhibitors). It is accordingly a primary object of this invention to provide an improved treatment in the form of a combination therapy, for at least one of obesity, cardiovascular diseases, and disorders of insulin metabolism, such as diabetes and syndrome X, using GDF-8 inhibitors in combination with at least one other therapeutic agent that treats the targeted syndromes.

One object of this invention is to create a method of treating at least one of obesity, cardiovascular diseases, and disorders of insulin metabolism in a subject, comprising administering to the subject a therapeutically effective amount of a GDF-8 inhibitor, and a therapeutically effective amount of at least one other therapeutic agent which treats the targeted syndrome.

A further object of this invention is to create a pharmaceutical composition for treating at least one of obesity, cardiovascular diseases, and disorders of insulin metabolism in a subject, comprising administering to the subject a therapeutically effective amount of a GDF-8 inhibitor, and a therapeutically effective amount of at least one other therapeutic agent which treats the targeted syndrome.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, and together with the description, serve to explain the principles of the invention.

### BRIEF DESCRIPTION OF THE SEQUENCES

TABLE 1
DNA and Amino Acid Sequences of Myo Fragments

| | Myo29 | Myo28 | Myo22 |
|---|---|---|---|
| DNA sequence of scFv | SEQ ID | SEQ ID | SEQ ID |
| | NO:4 | NO:5 | NO:6 |
| AA sequence of scFv | SEQ ID | SEQ ID | SEQ ID |
| | NO:7 | NO:8 | NO:9 |
| DNA sequence of VH | SEQ ID | SEQ ID | SEQ ID |
| | NO:10 | NO:11 | NO:12 |
| AA sequence of VH | SEQ ID | SEQ ID | SEQ ID |
| | NO:13 | NO:14 | NO:15 |
| DNA sequence of VL | SEQ ID | SEQ ID | SEQ ID |
| | NO:16 | NO:17 | NO:18 |
| AA sequence of VL | SEQ ID | SEQ ID | SEQ ID |
| | NO:19 | NO:20 | NO:21 |
| Germlined DNA seq. of | SEQ ID | SEQ ID | |
| scFv | NO:22 | NO:23 | |
| Germlined AA seq. of | SEQ ID | SEQ ID | |
| scFv | NO:24 | NO:25 | |
| Germlined DNA seq. VH | SEQ ID | SEQ ID | |
| | NO:26 | NO:27 | |
| Germlined AA seq. of VH | SEQ ID | SEQ ID | |
| | NO:28 | NO:29 | |
| Germlined DNA seq. of | SEQ ID | SEQ ID | |
| VL | NO:30 | NO:31 | |
| Germlined AA seq. of VL | SEQ ID | SEQ ID | |
| | NO:32 | NO:33 | |
| AA sequence of H1 | SEQ ID | SEQ ID | SEQ ID |
| | NO:34 | NO:35 | NO:36 |
| AA sequence of H2 | SEQ ID | SEQ ID | SEQ ID |
| | NO:37 | NO:38 | NO:39 |
| AA sequence of H3 | SEQ ID | SEQ ID | SEQ ID |
| | NO:40 | NO:41 | NO:42 |
| AA sequence of L1 | SEQ ID | SEQ ID | SEQ ID |
| | NO:43 | NO:44 | NO:45 |
| AA sequence of L2 | SEQ ID | SEQ ID | SEQ ID |
| | NO:46 | NO:47 | NO:48 |
| AA sequence of L3 | SEQ ID | SEQ ID | SEQ ID |
| | NO:49 | NO:50 | NO:51 |

TABLE 2
Sequence Chart

| | |
|---|---|
| AA sequence of mature GDF-8 | SEQ ID NO:1 |
| AA sequence of Myo-29 binding epitope | SEQ ID NO:2 |
| on GDF-8 containing variable Xaa | |
| positions | |
| AA sequence of specific Myo-29 binding | SEQ ID NO:3 |
| epitope on GDF-8 | |
| DNA sequence of C-terminal fragment of | SEQ ID NO:52 |
| IgG₁ light λ chain | |
| AA sequence of C-terminal fragment of | SEQ ID NO:53 |
| IgG₁ light λ chain | |
| DNA sequence of C-terminal fragment of | SEQ ID NO:54 |
| IgG₁ heavy λ chain | |
| AA sequence of C-terminal fragment of | SEQ ID NO:55 |
| IgG₁ heavy λ chain | |
| AA sequence of JA16 binding epitope on | SEQ ID NO:56 |
| any TGF-β family member. | |
| AA sequence of JA16 binding epitope on | SEQ ID NO:57 |
| GDF-8 | |
| AA sequence of JA16 binding epitope on | SEQ ID NO:58 |
| any TGF-β family member, longer | |
| AA sequence of JA16 binding epitope on | SEQ ID NO:59 |
| GDF-8, longer | |
| AA sequence of ActRIIB fusion protein | SEQ ID NO:60 |
| DNA sequence of ActRIIB fusion protein | SEQ ID NO:61 |
| AA sequence of ActRIIB | SEQ ID NO:62 |
| AA sequence of ActRIIB fusion protein | SEQ ID NO:63 |
| linker | |
| AA sequence of ActRIIB enterokinase | SEQ ID NO:64 |
| cleavage site | |
| GDF-8 propeptide | SEQ ID NO:65 |
| IgG1 Fc fragment | SEQ ID NO:66 |
| IgG1 modified for reduced effector | SEQ ID NO:67 |
| function Fc fragment | |
| Inhibitors of Proteases That Cleave GDF- | SEQ ID NO:68 |
| 8 Propeptide | |
| Inhibitors of Proteases That Cleave GDF- | SEQ ID NO:69 |
| 8 Propeptide | |
| Inhibitors of Proteases That Cleave GDF- | SEQ ID NO:70 |
| 8 Propeptide | |
| Inhibitors of Proteases That Cleave GDF- | SEQ ID NO:71 |
| 8 Propeptide | |
| Inhibitors of Proteases That Cleave GDF- | SEQ ID NO:72 |
| 8 Propeptide | |
| Inhibitors of Proteases That Cleave GDF- | SEQ ID NO:73 |
| 8 Propeptide | |
| Inhibitors of Proteases That Cleave GDF- | SEQ ID NO:74 |
| 8 Propeptide | |
| Inhibitors of Proteases That Cleave GDF- | SEQ ID NO:75 |
| 8 Propeptide | |
| Inhibitors of Proteases That Cleave GDF- | SEQ ID NO:76 |
| 8 Propeptide | |
| Inhibitors of Proteases That Cleave GDF- | SEQ ID NO:77 |
| 8 Propeptide | |

### DETAILED DESCRIPTION

I. Definitions

In order that the present invention may be more readily understood, certain terms are first defined. Additional definitions are set forth throughout the detailed description.

The term “antibody” refers to an immunoglobulin or fragment thereof, and encompasses any polypeptide comprising an antigen-binding site. The term includes but is not limited to polyclonal, monoclonal, monospecific, polyspecific, non-specific, humanized, human, single-chain, chimeric, synthetic, recombinant, hybrid, mutated, grafted, and in vitro generated antibodies. Unless preceded by the word “intact”, the term “antibody” includes antibody fragments such as Fab, F(ab′)₂, Fv, scFv, Fd, dAb, and other antibody fragments that retain antigen-binding function. Typically, such fragments would comprise an antigen-binding domain.

The term “effective amount” refers to a dosage or amount that is sufficient to ameliorate clinical symptoms of, or achieve a desired biological outcome in individuals suffering from at least one of obesity, cardiovascular diseases, and disorders of insulin metabolism, such as diabetes and syndrome X, using combination therapy.

The term “GDF-8” refers to a specific growth and differentiation factor-8 and, where appropriate, factors that are structurally or functionally related to GDF-8, for example, BMP-11 and other factors belonging to the TGF-β superfamily. The term refers to the full-length unprocessed precursor form of GDF-8 as well as the mature and propeptide forms resulting from post-translational cleavage. The term also refers to any fragments and variants of GDF-8 that maintain at least some biological activities associated with mature GDF-8, as discussed herein, including sequences that have been modified. The amino acid sequence of mature human GDF-8 is provided in SEQ ID NO:1. The present invention relates to GDF-8 from all vertebrate species, including, but not limited to, human, bovine, chicken, mouse, rat, porcine, ovine, turkey, baboon, and fish (for sequence information, see, e.g., McPherron et al., Proc. Nat. Acad. Sci. U.S.A. 94:12457-12461 (1997)).

The term “GDF-8 inhibitor” includes any agent capable of inhibiting activity, expression, processing, or secretion of GDF-8, or a pharmaceutically acceptable derivative thereof. Such inhibitors include GDF-8 inhibitors, such as antibodies against GDF-8 (such as Myo-29, Myo-28, Myo-22, and JA-16), antibodies against GDF-8 receptor, modified soluble receptors (including receptor fusions, such as the ActRIIB-Fc fusion), other proteins binding to GDF-8 (such as the GDF-8 propeptide, mutants of the GDF-8 propeptide, follistatin, follistatin-domain containing proteins, and Fc fusions of these proteins), proteins binding to the GDF-8 receptor and Fc fusions of these proteins, and mimetics of all the foregoing. Nonproteinaceous inhibitors (such as nucleic acids) are also encompassed by the term GDF-8 inhibitor. Such inhibitors are said to “inhibit”, “neutralize”, or “reduce” at least one of the physiologically growth-regulatory or morphogenetic activities associated with active GDF-8 protein. For example, GDF-8 can increase the level of blood sugar (hyperglycemia) or decrease weight or muscle mass. Also GDF-8 blocks insulin-induced expression of GLUT4, and it blocks insulin-mediated differentiation of pre-adipocytes. A reduction in activity may be about 10%, 20%, 30%, 40%, 50%, 60%, 70%, 80%, 90%, or more.

The term “specific binding”, such as when used in the context of a GDF-8 inhibitor, means that the inhibitor binds to at least one GDF-8 antigen. The term is also applicable where, e.g., an antigen binding domain of an antibody or other inhibitor is specific for a particular epitope, which is represented on a number of antigens, and the specific binding inhibitor carrying the antigen binding domain will be able to bind to the various antigens carrying the epitope. Typically, the binding is considered specific when the affinity constant Kₐ is higher than 10⁸ M⁻¹. An antibody or other inhibitor is said to “specifically bind” to an antigen if, under appropriately selected conditions, such binding is not substantially inhibited, while at the same time non-specific binding is inhibited.

The term “highly stringent” or “high stringency” describes conditions for hybridization and washing used for determining nucleic acid-nucleic acid interactions. Such conditions are known to those skilled in the art and can be found in, for example, “Current Protocols in Molecular Biology,” John Wiley & Sons, N.Y. 6.3.1-6.3.6 (1989). Both aqueous and nonaqueous conditions as described in the art can be used. One example of highly stringent hybridization conditions is hybridization in 6× sodium chloride/sodium citrate (SSC) at about 45° C., followed by at least one wash in 0.2×SSC, 0.1% SDS at 50° C. A second example of highly stringent hybridization conditions is hybridization in 6×SSC at about 45° C., followed by at least one wash in 0.2×SSC, 0.1% SDS at 55° C. Another example of highly stringent hybridization conditions is hybridization in 6×SSC at about 45° C., followed by at least one wash in 0.2×SSC, 0.1% SDS at 60° C. A further example of highly stringent hybridization conditions is hybridization in 6×SSC at about 45° C., followed by at least one wash in 0.2×SSC, 0.1% SDS at 65° C. Highly stringent conditions include hybridization in 0.5M sodium phosphate, 7% SDS at 65° C., followed by at least one wash at 0.2×SSC, 1% SDS at 65° C.

The phrase “moderately stringent” or “moderate stringency” hybridization refers to conditions that permit a nucleic acid to bind a complementary nucleic acid that has at least about 60%, at least about 75%, at least about 85%, identity to the nucleic acid; or at least about 90% identity to the nucleic acid Moderately stringent conditions comprise but are not limited to, for example, hybridization in 0.50% formamide, 5× Denhart's solution, 5×SSPE, 0.2% SDS at 42° C., followed by washing in 0.2×SSPE, 0.2% SDS, at 65° C. (see, e.g., Sambrook et al., Molecular Cloning A Laboratory Manual, Cold Spring Harbor Laboratory Press (1989)).

The phrase “substantially identical” or “substantially similar” means that the relevant amino acid or nucleotide sequence, such as of the GDF-8 inhibitors of the invention, will be identical to or have insubstantial differences (through conserved amino acid substitutions) in comparison to the sequences which are disclosed. Nucleotide and polypeptides of the invention include, for example, those that are at least about 50%, at least about 55%, at least about 60%, at least about 65%, at least about 70%, at least about 75%, at least about 80%, at least about 85%, at least about 90%, at least about 92%, at least about 93%, at least about 94%, at least about 95%, at least about 96%, at least about 97%, at least about 98%, or at least about 99% identical in sequence to nucleic acid molecules and polypeptides disclosed.

For polypeptides, at least 20, 30, 50, 100, or more amino acids will be compared between the original polypeptide and the variant polypeptide that is substantially identical to the original. For nucleic acids, at least 50, 100, 150, 300 or more nucleotides will be compared between the original nucleic acid and the variant nucleic acid that is substantially identical to the original. Thus, a variant could be substantially identical in a region or regions, but divergent in others, while still meeting the definition of “substantially identical.” Percent identity between two sequences is determined by standard alignment algorithms such as, for example, Basic Local Alignment Tool (BLAST) described in Altschul et al., J. Mol. Biol. 215:403-410 (1990), the algorithm of Needleman et al., J. Mol. Biol. 48:444-453 (1970), or the algorithm of Meyers et al., Comput. Appl. Biosci. 4:11-17 (1988).

The term “treatment” refers to a therapeutic or preventive measure. The treatment may be administered to a subject having a medical disorder or who ultimately may acquire the disorder, in order to prevent, cure, delay, reduce the severity of, or ameliorate one or more symptoms of a disorder or recurring disorder, or in order to prolong the survival of a subject beyond that expected in the absence of such treatment.

The term “targeted syndrome” refers to at least one of obesity, cardiovascular diseases, and disorders of insulin metabolism which is to be treated by the methods and combinations disclosed herein.

Examples of cardiovascular disorders include coronary artery disease (atherosclerosis), angina (including acute angina and unstable angina), heart attack, stroke (including ischemic stroke), hypertension associated cardiovascular diseases, coronary artery disease, hypertension, hyperlipidemia, peripheral arterial disease, and peripheral vascular disease. Examples of disorders of insulin metabolism include type 2 diabetes, syndrome X, impaired glucose tolerance, insulin resistance induced by trauma such as burns or nitrogen imbalance, metabolic syndrome, prediabetes, impaired glucose tolerance, and dyslipidemia.

The term “therapeutic agent” is a substance that treats or assists in treating a medical disorder.

As used herein, a “therapeutically effective amount” of a GDF-8 inhibitor and therapeutic agent refers to an amount which is effective, upon single or multiple dose administration to a subject (such as a human patient) at treating, preventing, curing, delaying reducing the severity of, ameliorating at least one symptom of a disorder or recurring disorder, or prolonging the survival of the subject beyond that expected in the absence of such treatment.

The term “variant” refers to nucleotide and amino acid sequences that are substantially identical or similar to the nucleotide and amino acid sequences of GDF-8 inhibitors (as well as GDF-8 itself) provided, respectively. Variants can be naturally occurring, for example, naturally occurring human and non-human nucleotide sequences, or be generated artificially. Examples of variants are those resulting from alternative splicing of the mRNA, including both 3′ and 5′ spliced variants, point mutations and other mutations, or proteolytic cleavage of the proteins. Variants include nucleic acid molecules or fragments thereof and amino acid sequences and fragments thereof, that are substantially identical or similar to other nucleic acids (or their complementary strands when they are optimally aligned (with appropriate insertions or deletions) or amino acid sequences respectively. In one embodiment, there is at least about 50% identity, at least about 55% identity, at least about 60% identity, at least about 65% identity, at least about 70% identity, at least about 75% identity, at least about 80% identity, at least about 85% identity, at least at least about 90%, at least about 92% identity, at least about 93% identity, at least about 94% identity, at least about 95% identity, at least about 96% identity, at least about 97% identity, at least about 98% identity, or at least about 99% identity between a nucleic acid molecule or protein of the invention and another nucleic acid molecule or protein respectively, when optimally aligned. Additionally, variants include proteins or polypeptides that exhibit GDF-8 activity or inhibit GDF-8 activity, as discussed in this application.

II. GDF-8 Inhibitors

GDF-8 inhibitors are useful in the treatment of obesity, cardiovascular diseases, and disorders of insulin metabolism, such diabetes and syndrome X. The use of these inhibitors is especially useful in the combination therapy of the present invention. GDF-8 inhibitors include antibodies (against GDF-8 and/or a GDF-8 receptor), modified soluble receptors, other proteins (including those that bind to GDF-8 and/or a GDF-8 receptor), propeptides, peptides and mimetics of all of these inhibitors. Nonproteinaceous inhibitors include, for example, nucleic acids.

Inhibitors that block the binding of GDF-8 to ActRIIB (a GDF-8 receptor) can be tested using an ActRIIB assay. GDF-8 may be biotinylated at a ratio of 20 moles of EZ-link Sulfo-NHS-Biotin (Pierce, Rockford, Ill., Cat. No. 21217) to 1 mole of the GDF-8 for 2 hours on ice. The reaction may be terminated by dropping the pH using 0.5% TFA and the complex may subjected to chromatography on a C₄ Jupiter 250×4.6 mm column (Phenomenex) to separate mature GDF-8 from GDF-8 propeptide. Biotinylated mature GDF-8 fractions eluted with a TFA/CH₃CN gradient were pooled, concentrated and quantified by MicroBCA protein Assay Reagent Kit (Pierce, Rockford, Ill., Cat. No. 23235).

Recombinant ActRIIB-Fc chimera (R&D Systems, Minneapolis, Minn., Cat. No. 339-RB/CF) may be coated on 96-well flat-bottom assay plates (Costar, N.Y., Cat. No. 3590) at 1 μg/ml in 0.2 M sodium carbonate buffer overnight at 4° C. Plates may be then blocked with 1 mg/ml bovine serum albumin and washed following standard ELISA protocol. 100 μl aliquots of biotinylated GDF-8 at various concentrations (such as 10 ng/ml) with or without a GDF-8 inhibitor (such as at concentrations ranging from 10⁻¹¹ M to 10⁻⁷ M) may be added to the blocked ELISA plate, incubated for 1 hr, washed, and the amount of bound GDF-8 detected by Streptavidin-Horseradish peroxidase (SA-HRP, BD PharMingen, San Diego, Calif., Cat. No. 13047E) followed by the addition of TMB (KPL, Gaithersburg, Md., Cat. No. 50-76-04). Colorimetric measurements may be done at 450 nM in a Molecular Devices microplate reader.

Inhibitors of the invention may also be tested using a reporter gene assay. See Thies et al., Growth Factors 18:251-259 (2001). For example, to demonstrate the activity of GDF-8, a reporter gene assay (RGA) has been developed using a reporter vector pGL3(CAGA)₁₂ expressing luciferase. The CAGA sequence was previously reported to be a TGF-β responsive sequence within the promoter of the TGF-β induced gene PAI-1 (Denner et al., EMBO J. 17:3091-3100 (1998)).

A reporter vector containing 12 CAGA boxes is made using the basic luciferase reporter plasmid pGL3 (Promega, Madison, Wis.). The TATA box and transcription initiation site from the adenovirus major late promoter (−35/+10) are inserted between the BglII and HindIII sites. Oligonucleotides containing 12 repeats of the CAGA boxes AGCCAGACA are annealed and cloned into the XhoI site. The human rhabdomyosarcoma cell line A204 (ATCC HTB-82) is then transiently transfected with pGL3(CAGA)₁₂ using FuGENE 6 transfection reagent (Boehringer Manheim, Germany). Following transfection, cells are cultured on 48 well plates in McCoy's 5A medium supplemented with 2 mM glutamine, 100 U/ml streptomycin, 100 μg/ml penicillin and 10% fetal calf serum for 16 hrs. Cells are then treated with or without 10 ng/ml GDF-8 and with or without the GDF-8 inhibitor at various concentrations for testing depending on the type of inhibitor in McCoy's 5A media with glutamine, streptomycin, penicillin, and 1 mg/ml bovine serum albumin for 6 hrs at 37° C. Inhibitor concentrations are selected from approximately 50 nM to 50 μM, for example. Exemplary concentrations include 1 nM, 10 nM, 50 nM, 100 nM, 500 nM, 1 μM, 5 μM, 10 μM, and 50 μM of GDF-8 inhibitor. Luciferase may be quantified in the treated cells using the Luciferase Assay System (Promega). Such an assay of GDF-8 activity will demonstrate whether a GDF-8 inhibitor is functioning effectively.

Animal-based testing can be used, such as in the obese Zucker diabetic rats described in Park et al., Circulation 104:815-819 (2001). The obese Zucker rat is characterized by excessive body weight, insulin resistance, hyperinsulinemia, and mild hyperglycemia, and is a well-established model of type 2 diabetes. Obese Zucker rats aged 8 to 9 weeks are used as the diabetic model, and lean Zucker rats aged 11 to 14 weeks are used as controls, for example. The combination therapy of the invention can be administered to the rats following the treatment plan sought to be evaluated. Investigators then track blood chemistry and morphology changes over time, for example, to assess effectiveness of a GDF-8 inhibitor.

A. GDF-8 Inhibitors

GDF-8 inhibitors that can block the activity of GDF-8 are useful in the invention. Such inhibitors may interact with GDF-8 itself. Alternatively, inhibitors may interact with a GDF-8 receptor (such as ActRIIB) or other binding partner, for example. Inhibitors may reduce or block the binding of GDF-8 to its receptor and/or the activity of the receptor after binding of GDF-8. Inhibitors, of course, may interact with both GDF-8 and a second factor, such as its receptor. In this regard, GDF-8 inhibitors include antibodies (against GDF-8 and/or a GDF-8 receptor), modified soluble receptors, other proteins (including those that bind to GDF-8 and/or a GDF-8 receptor), modified forms of GDF-8 or fragments thereof, propeptides, peptides, and mimetics of all of these inhibitors. Nonproteinaceous inhibitors include, for example, nucleic acids.

The GDF-8 inhibitors of the invention may be administered at a dosage from about 1 μg/kg to about 20 mg/kg, depending on the severity of the symptoms and the progression of the disease. The appropriate effective dose is selected by a treating clinician from the following ranges: about 1 μg/kg to about 20 mg/kg, about 1 μg/kg to about 10 mg/kg, about 1 μg/kg to about 1 mg/kg, about 10 μg/kg to about 1 mg/kg, about 10 μg/kg to about 100 μg/kg, about 100 μg to about 1 mg/kg, and about 500 μg/kg to about 1 mg/kg, for example. The GDF-8 inhibitors may be administered via topical, oral, intravenous, intraperitoneal, intramuscular, intracavity, subcutaneous or transdermal means.

It will be understood by one of ordinary skill in the art that certain amino acids in a sequence of any protein may be substituted for other amino acids without adversely affecting the activity of the protein. It is thus contemplated that various changes may be made in the amino acid sequences the sequence of the GDF-8 inhibitors of the invention, or DNA sequences encoding such GDF-8 inhibitors, without appreciable loss of their biological activity or utility. Such changes may include, but are not limited to, deletions, insertions, truncations, and substitutions.

The GDF-8 inhibitors are optionally glycosylated, pegylated, or linked to another nonproteinaceous polymer. The GDF-8 inhibitors of the invention may be modified to have an altered glycosylation pattern (i.e., altered from the original or native glycosylation pattern). As used herein, “altered” means having one or more carbohydrate moieties added or deleted, and/or having one or more glycosylation sites added or deleted as compared to the original inhibitor. Addition of glycosylation sites to the GDF-8 inhibitors may be accomplished by altering the amino acid sequence to contain glycosylation site consensus sequences well known in the art. Another means of increasing the number of carbohydrate moieties is by chemical or enzymatic coupling of glycosides to the amino acid residues of the inhibitor. These methods are described in WO 87/05330, and in Aplin et al., Crit. Rev. Biochem. 22:259-306 (1981). Removal of any carbohydrate moieties present on the receptor may be accomplished chemically or enzymatically as described by Sojar et al., Arch. Biochem. Biophys. 259:52-57 (1987); Edge et al., Anal. Biochem. 118:131-137 (1981); and by Thotakura et al., Meth. Enzymol. 138:350-359 (1987).

The GDF-8 inhibitors of the invention may also be tagged with a detectable or functional label. Detectable labels include radiolabels such as ¹³¹I or ⁹⁹Tc, which may be attached to GDF-8 inhibitors using conventional chemistry known in the art. Labels also include enzyme labels such as horseradish peroxidase or alkaline phosphatase. Labels further include chemical moieties such as biotin, which may be detected via binding to a specific cognate detectable moiety, e.g., labeled avidin.

1. Antibodies

Antibodies that inhibit GDF-8 activity are within the scope of the invention. Antibodies can be made, for example, by traditional hybridoma techniques (Kohler et al., Nature 256:495-499 (1975)), recombinant DNA methods (U.S. Pat. No. 4,816,567), or phage display techniques using antibody libraries (Clackson et al., Nature 352:624-628 (1991); Marks et al., J. Mol. Biol. 222:581-597 (1991)). For various other antibody production techniques, see, e.g., Antibodies: A Laboratory Manual, Eds. Harlow et al., Cold Spring Harbor Laboratory, (1988); and Antibody Engineering, 2nd ed., Oxford University Press, Ed. Borrebaeck, (1995). Antibodies may be fully or partly human or humanized. In certain embodiments, antibodies may have an altered or mutated Fc region as described in subsequent sections.

The affinity of antibodies for use in the combination therapies described herein may be between 10⁶ per mole and 10¹¹ per mole, and may be between 10⁸ per mole and 10¹⁰ per mole. In certain cases, the antibodies may inhibit GDF-8 activity in vitro and/or in vivo as demonstrated, for example, by inhibition of ActRIIB binding and reporter gene assays. The disclosed antibodies may inhibit GDF-8 activity associated with negative regulation of skeletal muscle mass and bone density. Antibodies to GDF-8 sequences are discussed in U.S. Pat. Nos. 5,827,733 and 6,096,506, for example.

a. Antibodies Against GDF-8

According to the methods described above, antibodies can be developed that bind to the GDF-8 protein itself. These antibodies will be effective in the invention if they inhibit an activity of GDF-8, for example if they block the binding of GDF-8 to its receptor. Antibodies that are most effective in this invention will have the property of binding specifically to GDF-8 or the GDF-8/GDF-8 receptor complex. Such antibodies may be capable of binding mature GDF-8 with high affinity, and may bind the mature protein in monomeric form, active dimer form, and/or as part of a GDF-8 latent complex.

i. Myo-29, Myo-28, and Myo-22

The Myo-29, Myo-28, and Myo-22 antibodies, described in further detail in U.S. Patent Pub. No. 2004/0142382-A1 (application Ser. No. 10/688,925), relevant portions of which are herein incorporated by reference, can be used in the methods of the invention. These antibodies are capable of binding mature GDF-8 with high affinity, inhibiting GDF-8 activity in vitro and in vivo as demonstrated, for example, by inhibition of ActRIIB binding and reporter gene assays, and inhibiting GDF-8 activity associated with negative regulation of skeletal muscle mass and bone density.

Exemplary DNA and amino acid (AA) sequences of Myo-29, Myo-28, and Myo-22 antibodies, their scFv fragments, V_{H} and V_{L} domains, and CDRs are set forth in the Sequences Listing and are enumerated as listed in Table 1. The sequences of heavy and light chains excluding the V_{H} and V_{L} domains are identical in Myo29, Myo28, and Myo22.

ii. JA-16

The JA-16 antibody, described in further detail in Whittemore et al., Bioch. Biophys. Res. Commun. 300:965-971 (2003), as well as in U.S. Patent Pub. No. 2003/0138422-A1 (application Ser. No. 10/253,532), relevant portions of both of which are herein incorporated by reference, binds to a mature GDF-8 protein as set forth in SEQ ID NO:1.

b. Antibodies Against GDF-8 Receptor

According to the methods described above, antibodies can be developed that bind to the GDF-8 receptor. These antibodies will be effective in the invention if they block the binding of GDF-8 to its receptor or if they block the activity of the receptor after binding of GDF-8. Antibodies can be developed against the whole receptor protein, or against only the extracellular domain. Antibodies may be developed against ActRIIB, ActRIIB variants, and other receptors for GDF-8 (see, e.g., U.S. Patent Pub. No. 2004/0223966-A1; U.S. Patent Pub. No. 2004/0077053-A1; WO 00/43781).

2. Modified Soluble Receptors

Modified soluble receptors of GDF-8 may be used in the invention. Soluble receptors may comprise all or part of the extracellular domain of a GDF-8 receptor, such as ActRIIB. The sequences of the ActRIIB receptor, including description of the extracellular domain, specific fragments and variants of the receptor are set forth in U.S. Pat. No. 6,656,475, for example. See, also, U.S. Pat. No. 6,696,260 and U.S. Patent Pub. No. 2004/0077053-A1 for further GDF-8 receptor structural and functional characteristics.

Such receptors may be produced recombinantly or by chemical or enzymatic cleavage of the intact receptor. The modified soluble receptors of the invention will bind GDF-8 in the blood stream, reducing the ability of GDF-8 to bind to the native GDF-8 receptor in the body. In such a way, these modified soluble receptors inhibit GDF-8 activity.

a. Receptor Fusions

The modified soluble receptors of the invention may be made more stable by fusion to another protein or portion of another protein. Increased stability is advantageous for therapeutics as they can be administered at a lower dose or at less frequent intervals. Fusion to at least a portion of an immunoglobulin, such as the constant region of an antibody, optionally an Fc fragment of an immunoglobulin, can increase the stability of a modified soluble receptor or other proteins of the invention. (See, e.g., Spiekermann et al., J. Exp. Med. 196:303-310 (2002)).

i. ActRIIB Fc Fusions

An ActRIIB Fc fusion inhibitor, described in further detail in U.S. Patent Pub. No. 2004/0223966-A1 (application Ser. No. 10/689,677), relevant portions of which are herein incorporated by reference, is comprised of a modified activin type II receptor ActRIIB that binds GDF-8 and inhibits its activity in vitro and in vivo. In particular, the ActRIIB fusion polypeptides inhibit the GDF-8 activity associated with negative regulation of skeletal muscle mass and bone density. ActRIIB fusion polypeptides described herein are soluble and possess pharmacokinetic properties that make them suitable for therapeutic use, e.g., extended circulatory half-life and/or improved protection from proteolytic degradation.

The ActRIIB fusion polypeptides to be used in compositions and methods of the invention comprise a first amino acid sequence derived from the extracellular domain of ActRIIB and a stabilizing portion or second amino acid sequence, such as a sequence derived from the constant region of an antibody. The full amino acid and DNA sequences of a particular illustrative embodiment of the ActRIIB fusion protein are set forth in SEQ ID NO:60 and SEQ ID NO:61, respectively.

The first amino acid sequence is derived from all or a portion of the ActRIIB extracellular domain and is capable of binding GDF-8 specifically. In some embodiments, such a portion of the ActRIIB extracellular domain may also bind BMP-11 and/or activin, or other growth factors. In certain embodiments, the first amino acid sequence is identical to or is substantially as set out in SEQ ID NO:60 from about amino acid (aa) 23 to about aa 138 or from about aa 19 to about aa 144 in SEQ ID NO:62. The difference between SEQ ID NO:62 and SEQ ID NO:60 is that aa 64 of SEQ ID NO:62 is Ala, whereas the corresponding aa 68 in SEQ ID NO:60 is Arg. Additionally, other variances in the sequence of ActRIIB are possible, for example, aa 16 and aa 17 in SEQ ID NO:62 can be substituted with Cys and Ala, respectively. In some other embodiments, the first amino acid sequence comprises at least 20, 30, 40, 50, 60, 70, 80, 90, 100, 110, or 120 contiguous amino acids from about aa 23 and about aa 138 of SEQ ID NO:60 or about aa 19 and about aa 144 of SEQ ID NO:62. Such a sequence can be truncated so long as the truncated sequence is capable of specifically binding GDF-8.

The second amino acid sequence is derived from the constant region of an antibody, particularly the Fc portion, or is a mutation of such a sequence. In some embodiments, the second amino acid sequence is derived from the Fc portion of an IgG. In related embodiments, the Fc portion is derived from IgG that is IgG₁, IgG₄, or another IgG isotype. In a particular embodiment, the second amino acid sequence comprises the Fc portion of human IgG₁ as set forth in SEQ ID NO:60 (amino acids 148 to 378), wherein the Fc portion of human IgG₁ has been modified to minimize the effector function of the Fc portion. Such modifications include changing specific amino acid residues which might alter an effector function such as Fc receptor binding (Lund et al., J. Immun. 147:2657-2662 (1991) and Morgan et al., Immunology 86:319-324 (1995)), or changing the species from which the constant region is derived. Antibodies may have mutations in the C_{H}2 region of the heavy chain that reduce effector function, i.e., Fc receptor binding and complement activation. For example, antibodies may have mutations such as those described in U.S. Pat. Nos. 5,624,821 and 5,648,260. In the IgG₁ or IgG₂ heavy chain, for example, such mutations may be made at amino acid residues corresponding to amino acids 234 and 237 in the full-length sequence of IgG₁ or IgG₂. Antibodies may also have mutations that stabilize the disulfide bond between the two heavy chains of an immunoglobulin, such as mutations in the hinge region of IgG₄, as disclosed in Angal et al., Mol. Immunol. 30:105-108 (1993).

In certain embodiments, the second amino acid sequence is linked to the C-terminus or the N-terminus of the first amino acid sequence, with or without being linked by a linker sequence. The exact length and sequence of the linker and its orientation relative to the linked sequences may vary. The linker may be, for example, (Gly-Ser)₂ (SEQ ID NO:63). The linker may comprise 2, 10, 20, 30, or more amino acids and is selected based on properties desired such as solubility, length and steric separation, immogenicity, etc. In certain embodiments, the linker may comprise a sequence of a proteolytic cleavage site, such as the enterokinase cleavage site Asp-Asp-Asp-Lys (SEQ ID NO:64), or other functional sequences useful, for example, for purification, detection, or modification of the fusion protein.

3. Other Proteins

Other proteins that inhibit GDF-8 activity may be used in the compositions and methods of the invention. Such proteins can interact with GDF-8 itself, inhibiting its activity or binding to its receptor. Alternatively, inhibitors can interact with a GDF-8 receptor (such as ActRIIB) and may be effective in compositions or methods if they block the binding of GDF-8 to its receptor or if they block the activity of the receptor after binding of GDF-8. Inhibitors, of course, may interact with both GDF-8 and its receptor. Inhibitors may also affect GDF-8 activity in other ways, such as by inhibiting the metalloprotease that cleaves the propeptide, which associates with mature GDF-8 and inhibits its activity (see, e.g., U.S. Patent Pub. No. 2004/0138118-A1).

a. Proteins Binding to GDF-8

Proteins that bind to GDF-8 and inhibit its activity (or binding to its receptor) are acceptable for use in the compositions and methods of the invention. While some proteins are known, additional proteins can be isolated using screening techniques, the ActRIIB binding assay, or reporter gene assays described above. Samples of proteins may be screened, as well as libraries of proteins.

i. GDF-8 Propeptide

GDF-8 propeptide can be used as an inhibitor of GDF-8. Because naturally occurring GDF-8 propeptides have a short in vivo half-life thereby reducing their effectiveness as pharmacological inhibitors of GDF-8 activity, a GDF-8 propeptide inhibitor includes modified and stabilized GDF-8 propeptides having improved pharmacokinetic properties, specifically an increased circulatory half-life. See U.S. Patent Pub. No. 2003/0104406-A1 (application Ser. No. 10/071,499), relevant portions of which are herein incorporated by reference.

Such modified GDF propeptides include fusion proteins comprising a GDF propeptide and an Fc region of an IgG molecule (as a stabilizing protein). These GDF inhibitors may comprise a GDF propeptide (for example as set forth in SEQ ID NO:5 or 11) or a fragment or variant of said propeptide which retains one or more biological activities of a GDF propeptide. The GDF-8 propeptides used in the invention may be synthetically produced, derived from naturally occurring (native) GDF-8 propeptides, or be produced recombinantly, using any of a variety of reagents, host cells and methods which are well known in the art of genetic engineering. In one embodiment, the modified GDF-8 propeptide comprises a human GDF-8 propeptide covalently linked to an IgG molecule or a fragment thereof. The GDF-8 propeptide may be linked directly to the Fc region of the IgG molecule, or linked to the Fc region of the IgG molecule via a linker peptide. Further proteins that bind to GDF-8, including propeptides of GDF-8 are provided in WO 00/43781.

iii. Foliistatin and Follistatin-Domain Containing Proteins

Proteins comprising at least one follistatin domain modulate the level or activity of growth and differentiation factor-8 (GDF-8), and may be used for treating disorders that are related to the modulation of the level or activity of GDF-8. Both follistatin itself and follistatin domain containing proteins (described in U.S. Patent Pub. Nos. 2003/0162714-A1 and 2003/0180306-A1 (application Ser. Nos. 10/369,736 and 10/369,738), relevant portions of both of which are herein incorporated by reference) may be used in the compositions and methods of the invention.

Proteins containing at least one follistatin domain will bind and inhibit GDF-8. Examples of proteins having at least one follistatin domain include, but are not limited to follistatin, follistatin-like related gene (FLRG), FRP (flik, tsc 36), agrins, osteonectin (SPARC, BM40), hevin (SC1, mast9, QR1), IGFBP7 (mac25), and U19878. GASP1 and GASP2 are other examples of proteins comprising at least one follistatin domain.

A follistatin domain, as stated above, is defined as an amino acid domain or a nucleotide domain encoding for an amino acid domain, characterized by cysteine rich repeats. A follistatin domain typically encompasses a 65-90 amino acid span and contains 10 conserved cysteine residues and a region similar to Kazal serine protease inhibitor domains. In general, the loop regions between the cysteine residues exhibit sequence variability in follistatin domains, but some conservation is evident. The loop between the fourth and fifth cysteines is usually small, containing only 1 or 2 amino acids. The amino acids in the loop between the seventh and eighth cysteines are generally the most highly conserved containing a consensus sequence of (G,A)-(S,N)-(S,N,T)-(D,N)-(G,N) followed by a (T,S)-Y motif. The region between the ninth and tenth cysteines generally contains a motif containing two hydrophobic residues (specifically V, I, or L) separated by another amino acid.

A follistatin domain-containing protein will comprise at least one, but possibly more than one, follistatin domain. The term also refers to any variants of such proteins (including fragments; proteins with substitution, addition or deletion mutations; and fusion proteins) that maintain the known biological activities associated with the native proteins, especially those pertaining to GDF-8 binding activity, including sequences that have been modified with conservative or non-conservative changes to the amino acid sequence. These proteins may be derived from any source, natural or synthetic. The protein may be human or derived from animal sources, including bovine, chicken, murine, rat, porcine, ovine, turkey, baboon, and fish.

Proteins comprising at least one follistatin domain, which may bind GDF-8, may be isolated using a variety of methods. For example, one may use affinity purification using GDF-8. In addition, one may use a low stringency screening of a cDNA library, or use degenerate PCR techniques using a probe directed toward a follistatin domain. As more genomic data becomes available, similarity searching using a number of sequence profiling and analysis programs, such as MotifSearch (Genetics Computer Group, Madison, Wis.), ProfileSearch (GCG), and BLAST (NCBI) could be used to find novel proteins containing significant homology with known follistatin domains.

One of skill in the art will recognize that GDF-8 or proteins comprising at least one follistatin domain, as well as other proteins described herein, may contain any number of conservative changes to their respective amino acid sequences without altering their biological properties. Such conservative amino acid modifications are based on the relative similarity of the amino acid side-chain substituents, for example, their hydrophobicity, hydrophilicity, charge, size, and the like. Exemplary conservative substitutions which take various of the foregoing characteristics into consideration are well known to those of skill in the art and include: arginine and lysine; glutamate and aspartate; serine and threonine; glutamine and asparagine; and valine, leucine, and isoleucine. Furthermore, proteins comprising at least one follistatin domain may be used to generate functional fragments comprising at least one follistatin domain. It is expected that such fragments would bind and inhibit GDF-8. In an embodiment of the invention, proteins comprising at least one follistatin domain specifically bind to mature GDF-8 or a fragment thereof, whether it is in monomeric form, active dimer form, or complexed in a GDF-8 latent complex, with an affinity of between 0.001 and 100 nM, or between 0.01 and 10 nM, or between 0.1 and 1 nM.

b. Proteins Binding to GDF-8 Receptor

Proteins that bind to a GDF-8 receptor (such as ActRIIB) and inhibit the binding of GDF-8 to the receptor or the activity of the receptor itself are acceptable for use within the scope of the invention. Such proteins can be isolated using screening techniques and the ActRIIB binding assay or reporter gene assays described above. Samples of proteins may be screened, as well as libraries of proteins.

c. Fusions with any of the Proteins Binding to GDF-8 or GDF-8 Receptor

Fusion proteins of any of the proteins that bind to GDF-8 or a GDF-8 receptor can be made more stable by fusion to another protein or portion of another protein. Increased stability is advantageous for therapeutics as they can be administered at a lower dose or at less frequent intervals. Fusion to at least a portion of an immunoglobulin, such as the constant region, optionally an Fc fragment of an immunoglobulin, can increase the stability of these proteins. The preparation of such fusion proteins is well known in the art and can be performed easily. (See, e.g., Spiekermann et al., J. Exp. Med., 196:303-310 (2002)).

A GDF-8 propeptide Fc fusion inhibitor, described in greater detail in U.S. Patent Pub. No. 2003/0104406-A1 (application Ser. No. 10/071,499), relevant portions of which are hereby incorporated by reference, comprises a polypeptide cleaved from the amino-terminal domain of the GDF-8 precursor protein, covalently linked with the Fc region of an IgG molecule or fragment thereof.

The GDF-8 propeptide Fc fusion inhibitor comprises a human GDF-8 propeptide or a mutant of GDF-8 propeptide, and the Fc region of an IgG₁ (SEQ ID NO:66), an IgG₄, or an IgG₁ modified for reduced effector function (SEQ ID NO:67). The GDF-8 propeptide may be modified to include stabilizing modifications.

Each of the GDF-8 propeptide inhibitors may be administered in therapeutically effective amounts. As used herein, an “effective amount” of the GDF-8 propeptide inhibitor is a dosage which is sufficient to reduce the activity of GDF-8 proteins to achieve a desired biological outcome, such as increasing skeletal muscle mass. Generally, a therapeutically-effective amount may vary with the subject's age, weight, physical condition, and sex, as well as the severity of the medical condition in the subject. The dosage may be determined by a physician and adjusted, as necessary, to suit observed effects of the treatment. The composition may be given at a dose from about 50 μg/kg to 20 mg/kg, such as from about 50 μg/kg to about 10 mg/kg, about 1 mg/kg to about 10 mg/kg, and about 5 mg/kg to about 10 mg/kg. The GDF-8 propeptide inhibitor may be given as a bolus dose, to maximize the circulating levels of GDF-8 propeptides for the greatest length of time after the dose. Continuous infusion may also be used after the bolus dose.

d. Inhibitors of Protease Activation of the GDF-8 Small Latent Complex

Inhibitors of protease activation of the GDF-8 small latent complex described in U.S. Patent Pub. No. 2004/0138118-A1 (application Ser. No. 10/662,438), relevant portions of which are incorporated herein by reference. Certain proteases cleave the propeptide, either in a free form or when it is associated with a mature GDF-8 dimer, rendering it unable to bind to and inhibit the activity of the mature GDF-8 dimer. As such, the proteases can convert a small latent complex (mature GDF-8 associated with and inhibited by propeptide) into active GDF-8. Once the propeptide has been cleaved it cannot bind to and inactivate the mature GDF-8 dimer. Inhibitors of protease activation of the GDF-8 small latent complex will enhance propeptide binding to mature GDF-8 dimers and inhibit GDF-8 activity. These inhibitors may competitively bind the protease, preventing it from binding the native small latent complex, or they may also bind the mature GDF-8 dimer creating an inhibitor-mature dimer complex, which is inactive and may optionally be resistant to protease cleavage.

The metalloproteases are exemplified by the BMP-1/TLD family of metalloproteases, which includes four mammalian proteins, BMP-1 (Wozney et al., Science 242:1528-1534 (1988)); mammalian Tolloid (mTLD) (Takahara et al., J. Biol. Chem. 269:32572-32578 (1994)); mammalian Tolloid-like-1 (mTLL-1) (Takahara et al., Genomics 34:157-165 (1996)); and mammalian Tolloid-like-2 (mTLL-2) (Scott et al., Devel. Biol. 213:283-300 (1999)), each of which is incorporated herein by reference.

The BMP-1/TLD family of metalloproteases, in turn, are members of a larger family of proteins, the astacin family, which includes proteases that are expressed in various vertebrate and invertebrate organisms, including, for example, Xenopus (Xolloid; UVS.2), fish (choriolysin H and L; zebrafish Tolloid), sea urchin (BP-10 and SpAN), and hydra (HMP-1; see, for example, Li et al., Proc. Natl. Acad. Sci., USA 93:5127-5130 (1996), which is incorporated herein by reference).

Inhibitors of protease activation of the GDF-8 small latent complex may be used for treatment of disorders according to this invention. Various metalloprotease inhibitors GDF-8 modulating agents are described in U.S. Patent Pub. No. 2004/0138118-A1, including antibody, nucleic acid, and peptide-based agents. Agents that inhibit the metalloprotease activity can include any type of molecule, including, for example, a peptide, peptide derivative such as a peptide hydroxamate or a phosphinic peptide, or peptoid and can be identified through the screening assays of U.S. Patent Pub. No. 2004/0138118-A1, for example. (See also, U.S. Patent Pub. No. 2005/0043232-A1).

Particular agents that inhibit protease activation of the GDF-8 small latent complex include peptides that compete for the metalloprotease enzyme with the propeptide GDF-8. These peptides can comprise a portion of the propeptide, a portion of the full length GDF-8 polypeptide containing the propeptide portion, or a derivative of a GDF-8 polypeptide having a mutation of a cleavage site for the metalloprotease. In one embodiment, a derivative of a peptide portion of GDF-8 is a peptide that corresponds to a GDF-8 propeptide. In one aspect of this embodiment, the derivative is a propeptide having a mutation of the metalloprotease cleavage site, for example, a substitution, deletion, or insertion of an amino acid at or in sufficient proximity to the cleavage site such that the metalloprotease has altered cleavage activity with respect to the peptide agent. In one aspect, agents that are resistant to metalloprotease cleavage inhibit or modulate metalloprotease mediated GDF-8 activation. In another aspect of this embodiment, a derivative of a peptide portion of GDF-8 is a peptide agent can contain one or more D-amino acids and/or L-amino acids; and/or one or more amino acid analogs, for example, an amino acid that has been derivatized or otherwise modified at its reactive side chain or its peptide linkage. Derivative or modified peptides can have improved stability to a protease, an oxidizing agent or other reactive material that the peptide may encounter in a biological environment.

The agent that modulates metalloprotease cleavage of the naturally occurring propeptide can be operatively linked to a second molecule, which facilitates the action or activity of the agent, alters the biological localization of the agent, or increases the stability of the agent in a particular environment. For example, a peptide agent can be stabilized by operatively linking the peptide agent to a polypeptide, such as a heterologous peptide. For example, it may be linked to an Fc domain of an antibody molecule, thereby increasing the half-life of the peptide agent in vivo.

Inhibitory antibodies against the metalloprotease enzymes can also be used in this invention and can easily be generated by known techniques in the art.

Peptide agents may be 10, 20, 30, 40, or 50 amino acid residues in length, containing wild type or mutant sequences, or derivatives thereof. For example, peptides having one or more amino acid changes at the P1 position (just upstream of the cleavage site) or the P1′ position (just downstream of the cleavage site) may be changed. An aspartic acid to alanine substitution at the P1′ position was tested in a series of peptides 10, 20, 30, 40 and 50 amino acids in length related to wild type GDF-8 propeptide sequence. Further, peptides having an arginine to glutamine substitution at the P1 position may be useful in vitro or in vivo inhibitors, as may wild type GDF-8 propeptide sequences. Specifically, alterations and derivative peptide agents having increased stability and/or resistance to protease cleavage are contemplated.

Individual peptide inhibitors of the metalloprotease enzymes include, but are not limited to:

(1) Peptides having aspartic acid to alanine substitutions at the P1′ position, such as:

(SEQ ID NO:68)

KDVIRQLLPKAPPLRELIDQYDVQRADSSDGSLEDDDYHATTET
IITMPT;

(SEQ ID NO:69)

QLLPKAPPLRELIDQYDVQRADSSDGSLEDDDYHATTETI;

(SEQ ID NO:70)

APPLRELIDQYDVQRADSSDGSLEDDDYHA;

(SEQ ID NO:71)

ELIDQYDVQRADSSDGSLED;
and

(SEQ ID NO:72)

YDVQRADSSD.

(2) Peptides having wild type metalloprotease cleavage sequences at the P1 and P1′ positions, such as:

(SEQ ID NO:73)

KDVIRQLLPKAPPLRELIDQYDVQRADSSDGSLEDDDYHATTET
IITMPT;

(SEQ ID NO:74)

QLLPKAPPLRELIDQYDVQRADSSDGSLEDDDYHATTETI

(SEQ ID NO:75)

APPLRELIDQYDVQRADSSDGSLEDDDYHA;

(SEQ ID NO:76)

ELIDQYDVQRADSSDGSLED;
and

(SEQ ID NO:77)

YDVQRADSSD.

4. Mimetics of GDF-8 Inhibitors

Mimetics of the GDF-8 inhibitors of the invention may be used. Any synthetic analogue of these GDF-8 inhibitors, especially those with improved in vitro characteristics such as having a longer half-life, or being less easily degraded by the digestive system, are useful.

Mimetics of antibodies against GDF-8, antibodies against GDF-8 receptor, modified soluble receptors and receptor fusions, and other proteins binding to GDF-8 such as GDF-8 propeptide, mutated GDF-8 propeptide, follistatin and follistatin-domain containing proteins, and Fc fusions thereof may all be used in the invention.

These mimetics will be effective in the invention if they block the activity of GDF-8, namely if they block the binding of GDF-8 to its receptor. Mimetics that are most effective in this invention will have the property of binding specifically to GDF-8 or the GDF-8/GDF-8 receptor complex. Such mimetics may be capable of binding mature GDF-8 with high affinity, and may bind the mature protein whether it is in monomeric form, active dimer form, or complexed in a GDF-8 latent complex. The mimetics of the invention may inhibit GDF-8 activity in vitro and in vivo as demonstrated, for example, by inhibition of ActRIIB binding and reporter gene assays. Further, the disclosed mimetics may inhibit GDF-8 activity associated with negative regulation of skeletal muscle mass and bone density.

B. Nonproteinaceous Inhibitors

Nonproteinaceous inhibitors include, for example, nucleic acids.

1. Nucleic Acids

The terms “polynucleotide,” “oligonucleotide,” and “nucleic acid” refer to deoxyribonucleic acid (DNA) and, where appropriate, to ribonucleic acid (RNA), or peptide nucleic acid (PNA). The term should also be understood to include nucleotide analogs, and single or double stranded polynucleotides (e.g., siRNA). Examples of polynucleotides include but are not limited to plasmid DNA or fragments thereof, viral DNA or RNA, antisense RNA, etc. The term “plasmid DNA” refers to double stranded DNA that is circular. “Antisense,” as used herein, refers to a nucleic acid capable of hybridizing to a portion of a coding and/or noncoding region of mRNA by virtue of sequence complementarity, thereby interfering with translation from the mRNA. The terms “siRNA” and “RNAi” refer to a nucleic acid which is a double stranded RNA that has the ability to induce degradation of mRNA thereby “silencing” gene expression. Typically, siRNA is at least 15-50 nucleotides long, e.g., 20, 21, 22, 23, 24, 25, 26, 27, 28, 29, or 30 nucleotides in length.

Nucleic acids that that can block the activity of GDF-8 are useful in this invention. Such inhibitors may encode proteins that interact with GDF-8 itself. Alternatively, such inhibitors may encode proteins that can interact with a GDF-8 receptor (such as ActRIIB) and may be effective in the invention if the encoded proteins block the binding of GDF-8 to its receptor or if they block the activity of the receptor after binding of GDF-8. Inhibitors, of course, may encode proteins that interact with both GDF-8 and its receptor. Such nucleic acids can be used to express GDF-8 inhibitors of the invention.

Alternatively, antisense nucleic acids may be used to inhibit the production of GDF-8 or a receptor of GDF-8 (such as ActRIIB). Antisense sequences can interact with complementary coding sequences to upset function, which may serve to inhibit GDF-8 or GDF-8 receptor production.

The nucleic acids for use in the invention may be identified using the ActRIIB binding assay and reporter gene assays described above.

The nucleic acids may be obtained, isolated, and/or purified from their natural environment, in substantially pure or homogeneous form. Systems for cloning and expression of a polypeptide in a variety of different host cells are well known. Suitable host cells include bacteria, mammalian cells, and yeast and baculovirus systems. Mammalian cell lines available in the art for expression of a heterologous polypeptide include Chinese hamster ovary cells, HeLa cells, baby hamster kidney cells, NSO mouse melanoma cells and many others. A common bacterial host is E. coli. For other cells suitable for producing proteins from nucleic acids see Gene Expression Systems, Eds. Fernandez et al., Academic Press (1999).

Suitable vectors can be chosen or constructed, containing appropriate regulatory sequences, including promoter sequences, terminator sequences, polyadenylation sequences, enhancer sequences, selection or marker genes and other sequences as appropriate. Vectors may be plasmids or viral, e.g., phage, or phagemid, as appropriate. For further details see, e.g., Molecular Cloning: A Laboratory Manual, Sambrook et al., 2nd ed., Cold Spring Harbor Laboratory Press (1989). Many known techniques and protocols for manipulation of nucleic acid, for example, in preparation of nucleic acid constructs, mutagenesis, sequencing, introduction of DNA into cells and gene expression, and analysis of proteins, are described in detail in Current Protocols in Molecular Biology, Eds. Ausubel et al., 2nd ed., John Wiley & Sons (1992).

A nucleic acid can be fused to other sequences encoding additional polypeptide sequences, for example, sequences that function as a marker or reporter. Examples of marker or reporter genes include β-lactamase, chloramphenicol acetyltransferase (CAT), adenosine deaminase (ADA), aminoglycoside phosphotransferase (responsible for neomycin (G418) resistance), dihydrofolate reductase (DHFR), hygromycin-B-phosphotransferase (HPH), thymidine kinase (TK), lacZ (encoding β-galactosidase), xanthine guanine phosphoribosyltransferase (XGPRT), luciferase, and many others known in the art.

The methods of the invention also encompass the use of short interfering RNAs (siRNA) and antisense oligonucleotides to reduce the expression of B7-H3 in order to enhance immune response. siRNA may be produced using standard techniques as described in Hannon, Nature 418:244-251 (2002); McManus et al., Nat. Reviews 3:737-747 (2002); Heasman, Dev. Biol. 243:209-214 (2002); Stein, J. Clin. Invest. 108:641-644 (2001); and Zamore, Nat. Struct. Biol., 8:746-750 (2001). Antisense nucleic acids may be produced using standard techniques as described in Antisense Drug Technology: Principles, Strategies, and Applications, 1st ed., Ed. Crooke, Marcel Dekker (2001).

Nucleic acids may be administered at a dosage from about 1 μg/kg to about 20 mg/kg, depending on the severity of the symptoms and the progression of the disease. The appropriate effective dose is selected by a treating clinician from the following ranges: about 1 μg/kg to about 20 mg/kg, about 1 μg/kg to about 10 mg/kg, about 1 μg/kg to about 1 mg/kg, about 10 μg/kg to about 1 mg/kg, about 10 μg/kg to about 100 μg/kg, about 100 μg to about 1 mg/kg, and about 500 μg/kg to about 1 mg/kg. Nucleic acid inhibitors may be administered via topical, oral, intravenous, intraperitoneal, intramuscular, intracavity, subcutaneous or transdermal means.

III. Other Therapeutic Agents for Use in Combination with GDF-8 Inhibitors

A. Insulin

Insulins useful with the methods and combinations of this invention include rapid acting insulins, intermediate acting insulins, long acting insulins and combinations of intermediate and rapid acting insulins. Insulin therapy replaces insulin that is not being produced by the body. The combination of a rapid- or short-acting and intermediate- or long-acting insulin helps keep blood sugar levels within normal or closer to normal levels. The use of these agents is described in further detail in U.S. Patent Pub. No. 2002/0187980-A1 (application Ser. No. 10/164,235), relevant portions thereof are herein incorporated by reference.

Rapid acting commercially available insulin products include the HUMALOG® Brand Lispro Injection (rDNA origin), HUMULIN® R Regular Human Injection, USP [rDNA origin], HUMULIN® R Regular U-500 Concentrated Human Injection, USP [rDNA origin], REGULAR ILETIN® II (insulin injection, USP, purified pork) available from Eli Lilly and Co., and the NOVOLIN® Human Insulin Injection and VENOSULIN® BR Buffered Regular Human Injection, each available from Novo Nordisk Pharmaceuticals.

Commercially available intermediate acting insulins useful with this invention include, but are not limited to, the HUMULIN® L brand LENTE® human insulin (recombinant DNA origin) zinc suspension, HUMULIN® N NPH human insulin (recombinant DNA origin) isophane suspension, LENTE® ILETIN® II insulin zinc suspension, USP, purified pork, and NPH ILETIN® II isophane insulin suspension, USP, purified pork, available from Eli Lilly and Company, LANTUS® insulin glargine (recombinant DNA origin) injection, available from Aventis Pharmaceuticals, and the NOVOLIN L Lente® human insulin zinc suspension (recombinant DNA origin), and NOVOLIN® N NPH human insulin isophane suspension (recombinant DNA origin) products available from Novo Nordisk Pharmaceuticals, Inc, Princeton N.J.

Also useful with the methods and formulations of this invention are intermediate and rapid acting insulin combinations, such as the HUMALOG® Mix 75/25™ (75% Insulin Lispro Protamine Suspension and 25% Insulin Lispro Injection), HUMULIN® 50/50 (50% Human Insulin Isophane Suspension and 50% Human Insulin Injection) and HUMULIN® 70/30® (70% Human Insulin Isophane Suspension and 30% Human Insulin Injection), each available from Eli Lilly and Company. Also useful are the NOVALIN® 70/30 (70% NPH, Human Insulin Isophane Suspension and 30% Regular, Human Insulin Injection) line of combination products, which are intermediate and rapid acting insulin available from Novo Nordisk Pharmaceuticals.

An exemplary commercially available long acting insulin for use with this invention is the HUMULIN® U Ultralente® human insulin (recombinant DNA origin) extended zinc suspension, available from Eli Lilly and Company.

Also useful in the methods of this invention are inhaled insulin products, such as the EXUBERA® inhaled insulin product developed by Pfizer Inc. and Aventis SA.

Each of these insulin products can be administered as directed by a medical professional using administrations, dosages and regimens known in the art, such as those published for each product in the Physicians' Desk Reference, 55 Edition, 2001, published by Medical Economics Company, Inc. at Montvale, N.J., the relevant sections of which are incorporated herein by reference.

B. Sulfonylurea Agents

Sulfonylurea agents increase the amount of insulin produced by the pancreas. They also increase the effectiveness of insulin throughout the body by increasing functionality of insulin receptors and stimulating the production of more insulin receptors. These agents also reduce insulin resistance and may reduce the amount of sugar made by the liver.

Sulfonylurea agents useful with the methods and compositions of this invention include glipizide, glyburide (glibenclamide), chlorpropamide, tolbutamide, tolazamide and glimepriride, or the pharmaceutically acceptable salt forms thereof. The use of these agents are described in further detail in U.S. Patent Pub. No. 2003/008869-A1 (application Ser. No. 10/163,783), relevant portions of which are herein incorporated by reference.

The sulfonylurea agents of this invention may be administered at doses and regimens known in the art, such as those listed for the relevant compounds in the Physicians' Desk Reference, 55 Edition, 2001, published by Medical Economics Company, Inc. at Montvale, N.J. For example, glimepiride, which is available in AMARYL® tablets from Aventis Pharmaceuticals, may be given at an initial daily dosage of from about 1 to about 2 mg per day in human adults. This dosage may be increased gradually up to about 8 mg per day, with a usual maintenance dose being between about 2 and 4 mg per day. Glyburide is available in DIAβETA® tablets from Aventis Pharmaceuticals, and has an initial dose ranging from about 2.5 to about 5 mg per day and a usual maintenance dose of from about 1.25 to about 20 mg per day. Chlorpropamide is available from Pfizer Inc. in DIABINESE® tablets, and may have a daily dose in humans of from about 100 to about 500 mg, depending upon the individual characteristics of the recipient. Glipizide is commercially available in GLUCOTROL® tablets and GLUCOTROL XL® extended release tablets from Pfizer Inc. It can be administered at an initial daily dose of from about 2.5 to about 5 mg and increased in 2.5 to 5 mg increments to a maintenance dose of between about 15 and 40 mg per day. Tolazamide is generally administered at a daily dosage of between about 100 mg and 500 mg per day, with an average maintenance dose of between about 250 mg and 500 mg per day taken once daily or divided into multiple administrations over the course of a day. 250 mg and 500 mg tablets of tolazamide and 500 mg tablets of tolbutamide are available from Mylan Pharmaceuticals Inc., Morgantown, W. Va., U.S.A.

C. Biguanide Agents

Biguanide agents lower blood sugar by decreasing the amount of sugar produced by the liver in gluconeogenesis. They also increase the amount of sugar absorbed by muscle cells and decrease insulin resistance. These agents may lower triglyceride levels in the blood and reduce certain abnormal clotting factors and markers of inflammation that can lead to atherosclerosis.

Biguanide agents useful with the methods and compositions of this invention include mefformin and its pharmaceutically acceptable salt forms. The use of these agents is described in further detail in U.S. Patent Pub. No. 2003/0018028-A1 (application Ser. No. 10/163,707), relevant portions thereof are herein incorporated by reference.

Metformin hydrochloride useful in the methods and combinations is commercially available in 500 mg, 850 mg and 1000 mg tablets under the GLUCOPHAGE® tradename from Bristol Myers Squibb. Metformin hydrochloride may be administered in humans at an initial daily dose of from 500 mg to about 800 mg and increased, as needed, to a maximum daily dosage of 2550 mg.

D. Thiazolidinedione Agents

Thiazolidinedione agents improve the way cells in the body respond to insulin by lowering insulin resistance. They also may help in the treatment of high cholesterol by reducing triglycerides and increasing high-density lipoproteins (HDL) in the blood.

Thiazolidinedione agents useful with the methods and compositions of this invention are the non-limiting group of pioglitazone or rosiglitazone, or a pharmaceutically acceptable salt form of these agents. The use of these agents is described in further detail in U.S. Patent Pub. No. 2002/0198203-A1 (application Ser. No. 10/164,233), relevant portions thereof are herein incorporated by reference. Each of these agents may be produced by methods known in the art. These agents may also be administered at the pharmaceutically or therapeutically effective dosages or amounts known in the art for these compounds, such as those described in the Physician's Desk Reference 2001, 55 Edition, Copyright 2001, published by Medical Economics Company, Inc., the relevant portions describing each of these products being incorporated herein by reference.

Pioglitazone is available in the form of 15 mg, 30 mg and 45 mg ACTOS® brand pioglitazone hydrochloride tablets from Swiss Bioceutical International, Ltd. Pioglitazone and its pharmaceutically acceptable salt forms may be administered in humans at an initial daily dose of from about 15 mg or 30 mg and increased, as needed, to a maximum daily dose of about 45 mg.

Rosiglitazone is available in the form of 2 mg, 4 mg and 8 mg AVANDIA® rosiglitazone maleate tablets from GlaxoSmithKline. Rosiglitazone may be administered in humans at an initial daily dose of about 4 mg in a single or divided doses and increased, as needed, up to a maximum daily dose of 8 mg.

E. Alpha-Glucosidase Inhibitors

Alpha-glucosidase inhibitors delay the digestion of carbohydrates in the body and slow the rate at which the intestines absorb glucose from food. This decreases the amount of sugar that passes into your blood after a meal and prevents periods of hyperglycemia.

Alpha-glucosidase inhibitors which may be used with the methods and compositions of the invention described herein are miglitol or acarbose, or a pharmaceutically acceptable salt form of one or more of these compounds. The use of these agents is described in further detail in U.S. Patent Pub. No. 2003/0013709-A1 (application Ser. No. 10/164,232), relevant portions thereof are herein incorporated by reference.

Acarbose tablets are available from Bayer Corporation under the PRECOSE® tradename, which may be administered in humans at an initial dose of about 25 mg administered from one to three times daily and increased over time to a range of from about 50 to 100 mg administered three times per day.

Miglitol tablets in 25 mg, 50 mg and 100 mg doses are available under the GLYSET™ tradename from Pharmacia & Upjohn and may be administered at an initial dose of about 25 mg per day and increased as needed to a maximum dose of 100 mg administered three times daily.

F. PTPase Inhibitors

Protein tyrosine phosphatases (PTPases) are a large family of diverse molecules that can play an important role in modulating a wide variety of cellular responses. The PTPase family is divided into three major subclasses, classical PTPases, low molecular weight PTPases, and dual specificity PTPases. The classical PTPases can be further categorized into two classes, intracellular PTPases (e.g., PTP1B, TC-PTP, rat-brain PTPase, STEP, PTPMEG1, PTPH1, PTPD1, PTPD2, FAP-1/BAS, PTP1C/SH-PTP1/SHP-1 and PTP1D/Syp/SH-PTP2/SHP2) and receptor-type PTPases (e.g., CD45, LAR, PTP1, PTP1, PTPA, PTPM, PTPK, SAP-1 and DEP-1). Dual specificity phosphatases have the ability to remove the phosphate group from both serine/threonine and tyrosine residues. Members of the PTPase family have been implicated as important modulators or regulators of a wide variety of cellular processes including insulin signaling, leptin signaling, T-cell activation and T-cell mediated signaling cascade, the growth of fibroblasts, platelet aggregation, and regulation of osteoblast proliferation.

Certain PTPase inhibitors are described in detail in U.S. Patent Application Nos. 60/547,071 and 60/547,049, relevant portions of which are herein incorporated by reference. Other PTPase inhibitors may be used in this invention as well.

In one aspect, a PTPase inhibitor has the formula (I): 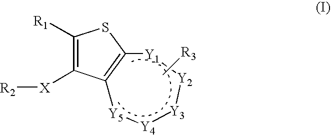
R₁ is C(O)OR₇, 5- to 6-membered heterocycle, H, halogen, CN, or C(O)NR₇R₈.
R₂ is C(O)ZR₄ or CN.
Z is —O— or —NR₅—.
X is —O—C₁₋₃alkylene-, —NR₈—C₁₋₃alkylene-, —S—C₁₋₃alkylene-, —SO—C₁₋₃alkylene-, —SO₂—C₁₋₃alkylene-, —C₁₋₄alkylene-, —C₂₋₄alkenylene-, or —C₂₋₄alkynylene-. Any of the alkylene, alkenylene and alkynylene groups can be optionally substituted with one or more halogen, oxo, HN═, CN, OCF₃, OH, NH₂, NO₂, R₄, or Q.
Each Y₁, Y₂, Y₃, Y₄, and Y₅ is, independently, CR₃, N, S, or O. One or two of Y₁, Y₂, Y₃, Y₄, and Y₅ can be absent.
Each R₃ is, independently, H, aryl, 5- to 8-membered heterocyclyl, C₁₋₆alkyl, C₂₋₆alkenyl, C₂₋₆alkynyl, halogen, CN, OCF₃, OH, NH₂, NO₂, or Q. Any of the aryl, heterocyclic, alkyl, alkenyl or alkynyl groups is optionally substituted with one or more halogen, oxo, CN, OCF₃, OH, NH₂, NO₂, N₃, R₄, or Q.
Each Q is, independently, —OC(O)NR₄R₅, —OR₄, —OC(O)R₄, —COOR₄, —C(O)NR₄R₅, —C(O)R₄, —C(═N—OH)R₄, —NR₄R₅, —N⁺R₄R₅R₆, —NR₄C(O)R₅, —NR₄C(O)NR₅R₆, —NR₄C(O)OR₅, —NR₄S(O)₂R₅, —SR₄, —S(O)R₄, —S(O)₂R₄, or —S(O)₂NR₄R₅.

Each R₄, R₅, and R₆ is, independently, H, C₁₋₁₆alkyl, C₂₋₁₂alkenyl, C₂₋₁₂alkynyl, C₃₋₈cycloalkyl, cycloalkylC₁₋₆alkyl, 5- to 8-membered heterocycle, heterocyclicC₁₋₆alkyl, aryl, arylC₁₋₆alkyl, arylC₂₋₆alkenyl, or arylC₂₋₆alkynyl. Each R₄, R₅, and R₆ can be optionally substituted with one or more C₁₋₆alkyl, C₂₋₆alkenyl, C₂₋₆alkynyl, halogen, oxo, CN, OCF₃, OH, NH₂, NO₂, N₃, —OC(O)NR₇R₈, —OR₇, —OC(O)R₇, —COOR₇, —C(O)NR₇R₈, —C(O)R₇, —NR₇R₈, —N⁺R₇R₈R₉, —NR₇C(O)R₈, —NR₇C(O)NR₈R₉, —NR₇C(O)OR₈, —NR₇S(O)₂R₈, —SR₇, —S(O)R₇, —S(O)₂R₇, or —S(O)₂NR₇R₈.

Each R₇, R₈, and R₉ is, independently, H, C₁₋₁₂alkyl, C₂₋₁₂alkenyl, C₂₋₁₂alkynyl, C₃₋₁₂cycloalkyl, aryl, or arylC₁₋₁₂alkyl. Each R₇, R₈, and R₉ can be optionally substituted with one or more halogen, oxo, CN, OCF₃, OH, NH₂, or NO₂.

When the ring system is 1-benzothiophene, R₁ is C(O)OCH₃, and X is —OCH₂—, then R₂ is not C(O)OCH₃.
When the ring system is 1-benzothiophene, R, is C(O)OH, and X is —OCH₂—, then R₂ is not C(O)OH.
When the ring system is thieno[2,3-b]pyridine, R, is isopropyl ester, and X is —OCH₂—, then R₂ is not C₁₋₃alkyl ester.
When the ring system is thieno[2,3-b]pyridine, R₁ is C(O)OC₁₋₄alkyl, and X is —OCH₂— or —OCH(CH₃)—, then R₂ is not CN.
When the ring system is thieno[2,3-b]pyridine, R₁ is isopropyl ester, and X is —SCH₂CH₂—, then R₂ is not CN.
When the ring system is thieno[2,3-b]pyridine, R₁ is isopropyl ester, and X is —SCH₂—, then R₂ is not isopropyl ester.

In certain embodiments, R₁ is a 5- or 6-membered heterocycle. Preferred 5-membered heterocycles can include the following:

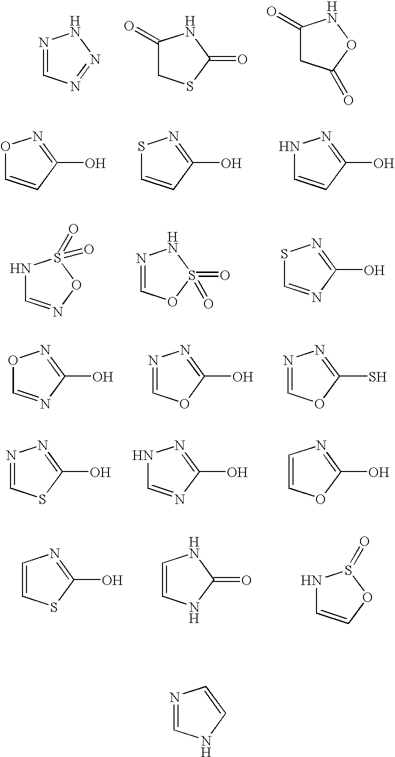

In certain embodiments, R₁ and R₂ are —C(O)OH or —C(O)OC₁₋₄alkyl. In another aspect, X is —O—C₁₋₃alkylene-, —NR₈—C₁₋₃alkylene-, —S—C₁₋₃alkylene-, —SO—C₁₋₃alkylene-, or —SO₂—C₁₋₃alkylene-, wherein any alkylene group is optionally substituted with one or more F, Cl, CN, OCF₃, OH, NH₂, NO₂, CHO, or Q. In certain embodiments, X is —O—CH₂—. In another aspect, the fused heterocycle is benzothiophene or thienopyridine.

The compound of formula (I) can be a salt. It may also be included in a pharmaceutical composition as a pharmaceutically acceptable salt or prodrug thereof, in combination with a pharmaceutically acceptable excipient or carrier. The compound can inhibit a PTPase such as PTP1B.

In another embodiment of the invention, the PTPase inhibitor may also be a compound having the formula (II): 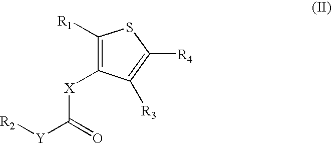
R₁ is R₅, OR₅, C(O)OR₅, C(O)R₅, or C(O)NR₅R₆.
R₂ is R₅.
X is —O—C₁₋₃alkylene-, —NR₈—C₁₋₃alkylene-, —S—C₁₋₃alkylene-, —SO—C₁₋₃alkylene-, —SO₂—C₁₋₃alkylene-, —C₁₋₄alkylene-, —C₂₋₄alkenylene-, or —C₂₋₄alkynylene-. Any of the alkylene, alkenylene or alkynylene groups can be optionally substituted with one or more halogen, oxo, imido, CN, OCF₃, OH, NH₂, NO₂, or Q.
Y is absent, —O—, or —NR₆—.
R₃ is H, halogen, CN, CF₃, OCF₃, C₁₋₃ alkyl, C₃₋₄cycloalkyl, C₁₋₃alkoxy, or aryl.
R₄ is A-B-E-D, where A is absent or arylene, heteroarylene, C₁₋₆alkylene, C₂₋₆ alkenyldiyl, or C₂₋₆alkynyl. Each A can be optionally substituted with one or more of C₁₋₆alkyl, C₂₋₆alkenyl, C₂₋₆alkynyl, halogen, CN, OCF₃, OH, NH₂, CHO, NO₂, or Q. Any of the alkyl, alkenyl or alkynyl groups is optionally substituted with one or more halogen, oxo, CN, OCF₃, OH, NH₂, NO₂, N₃, or Q. Each A can be optionally terminated with one or more arylene, alkylene, or alkenylene.
B is absent or —NR₅—, —NR₇—, —N(R₅)CH₂—, —N(R₇)CH₂—, —N(R₉)—, —N(R₉)C(O)—, —N(R₉)C(O)C(R₁₁)(R₁₂)—, —N(R₉)C(O)C(O)—, —N(R₉)C(O)N(R₁₀)—, —N(R₉)SO₂—, —N(R₉)SO₂C(R₁₀)(R₁₁)—, —N(R₉)(R₁₀)C(R₁₁)(R₁₂)—, —N(R₉)C(R₁₁)(R₁₂)C(R₁₃)(R₁₄)—, —O—, —O—C(R₁₁)(R₁₂), —O—C(R₁₁)(R₁₂)C(R₁₃)(R₁₄)—, —C(R₁₁)(R₁₂)—O—, —C(R₁₁)(R₁₂)—O—C(R₁₃)(R₁₄)—, —C(R₁₁)(R₁₂)N(R₉)—, —C(R₁₁)(R₁₂)N(R₉)C(R₁₃)(R₁₄)—, —C(R₁₁)(R₁₂)S—, —C(R₁₁)(R₁₂)SC(R₁₃)(R₁₄)—, or —C(R₁₁)(R₁₂)SO₂C(R₁₃)(R₁₄)—.
E is absent or C₃₋₁₂cycloalkylene, 3- to 12-membered heterocycdiyl, arylene, C₁₋₁₂alkylene, C₂₋₁₂alkenylene, or C₂₋₁₂alkynylene, where each E is optionally substituted with one or more C₁₋₃alkyl, C₁₋₃alkoxy, halogen, CN, OH, NH₂, or NO₂.
D is one or more H, halogen, OH, NH₂, CHO, CN, NO₂, CF₃, or Q.
When A, B, and E are absent, R₁ is C(O)OH or C(O)OCH₃, R₂ is H, and R₃ is H or chlorine, D is not H or chlorine; and when A, B, and E are absent, R₁ is C(O)OH or C(O)OCH₃, R₂ is H, and R₃ is H or bromine, D is not H or bromine.
Each Q, independently, is —R₅, —R₇, —OR₅, —OR₇, —NR₅R₆, —NR₅R₇, —N⁺R₅R₆R₈, S(O)ₙR₅, or —S(O)ₙR₇, and n is 0, 1, or 2.
Each R₅, R₆, and R₈, independently, is H, C₁₋₁₂alkyl, C₂₋₁₂alkenyl, C₂₋₁₂alkynyl, C₃₋₁₂cycloalkyl, C₁₋₁₂alkoxyC₁₋₁₂alkyl, cycloalkylC₁₋₆alkyl, 3- to 8-membered heterocycyl, heterocycylC₁₋₆alkyl, aryl, arylC₁₋₆ alkyl, arylC₂₋₆ alkenyl, or arylC₂₋₆ alkynyl. Each R₅, R₆, and R₈ can be optionally substituted with one or more R₉, —OR₉, —OC(O)OR₉, —C(O)R₉, —C(O)OR₉, —C(O)NR₉R₁₀, —SR₉, —S(O)R₉, —S(O)₂R₉, —NR₉R₁₀, —N⁺R₉R₁₀R₁₁, —NR₉C(O)R₁₀, —NC(O)NR₉R₁₀, —NR₉S(O)₂R₁₀, oxo, halogen, CN, OCF₃, CF₃, OH, or NO₂.
R₇ is —C(O)R₅, —C(O)OR₅, —C(O)NR₅R₆, —S(O)₂R₅, —S(O)R₅, or —S(O)₂NR₅R₆.
Each R₉, R₁₀, R₁₁, R₁₂, R₁₃ and R₁₄ is, independently, H, C₁₋₁₂alkyl, C₂₋₁₂alkenyl, C₂₋₁₂alkynyl, C₃₋₁₂cycloalkyl, aryl, or arylC₁₋₁₂alkyl. Any of the alkyl, alkenyl, alkynyl, cycloalkyl, aryl, or arylalkyl groups is optionally substituted with one or more halogen, oxo, CN, OCF₃, OH, NH₂, or NO₂.

In certain embodiments, R₁ is C(O)OH, C(O)OCH₃, C(O)OCH₂CH₃, or C(O)NH₂. In other embodiments, R₂ is H, CH₃, CH₂CH₃, or t-butyl. In certain embodiments, X is —O—C₁₋₃alkyl-, —N—C₁₋₃alkyl-, —S—C₁₋₃alkyl-, —SO—C₁₋₃alkyl-, or —SO₂—C₁₋₃alkyl-. In other embodiments, R₃ is H, F, Cl, Br, methyl, or CF₃.

In one embodiment, A is an aryl group substituted with B and may furthermore be optionally substituted with one or more of OH, NH₂, CHO, CN, NO₂, halogen, C₁-C₄ alkyl or Q.; B can be absent or a 1-3 atom linker such as C₁-C₃ alkyl, C₂-C₃ alkenyl, NH, NHCO, NHCONH, NHSO₂, NHSO₂CH₂, NHCH₂, NHCH₂CH₂, O, OCH₂, OCH₂CH₂, CH₂O, CH₂OCH₂, CH₂NH, CH₂NHCH₂, CH₂S, CH₂SCH₂, or CH₂SO₂CH₂.

In the following examples, for the connection of B-E-D to A, it is shown that the meta positions (C-3 or C-5) relative to the connection between A and the thiophene ring are preferred when A is a 6-membered aryl group. When A is a 5-membered aryl group, the C-3 or C-4 positions relative to the connection between A and the thiophene ring are preferred.

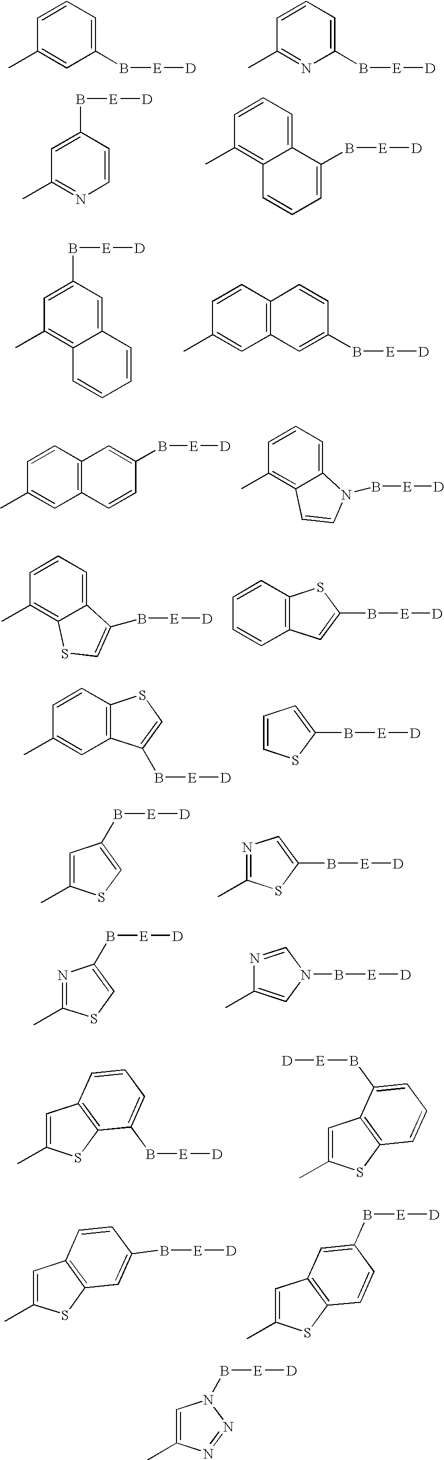

In another embodiment, E is absent or C₃₋₈cycloalkylene, C₃₋₈heterocycdiyl, arylene, C₁₋₆alkylene, C₂₋₆alkenylene, or C₂₋₆alkynylene, and is optionally substituted with one or more C₁₋₃alkyl, C₁₋₃alkoxy, halogen, CN, OH, NH₂, or NO₂. In certain embodiments, E can be cyclopentdiyl, cyclohexdiyl, cycloheptdiyl, piperidindiyl, piperazindiyl, pyrrolidindiyl, tetrahydrofurandiyl, morpholindiyl, phenylene, pyridindiyl, pyrimidindiyl, thiophendiyl, furandiyl, imidazoldiyl, pyrroldiyl, benzimidazoldiyl, tetrahydrothiopyrandiyl, or tetrahydropyrandiyl.

In one embodiment, D is one or more H, halogen, OH, NH₂, CHO, CN, NO₂, CF₃, aryl, or Q. In certain embodiments, D is SO₂R₇, —C(O)R₇, —OC(O)NR₅R₆, —OR₇, —COOR₇, —C(O)NR₅R₆, —C(O)R₇, pyrimidinyl or pyridinyl.

The compound of formula (II) can be a salt. It may also be included in a pharmaceutical composition as a pharmaceutically acceptable salt or prodrug thereof, in combination with a pharmaceutically acceptable excipient or carrier. The compound can inhibit a PTPase such as PTP1B.

Effective administration of these compounds may be given at a daily dosage of from about 1 mg/kg to about 250 mg/kg, for example, and may be given in a single dose or in two or more divided doses. Such doses may be administered in any manner useful in directing the active compounds herein to the recipient's bloodstream, including orally, via implants, parenterally (including intravenous, intraperitoneal and subcutaneous injections), rectally, vaginally, and transdermally. For the purposes of this disclosure, transdermal administrations are understood to include all administrations across the surface of the body and the inner linings of bodily passages including epithelial and mucosal tissues. Such administrations may be carried out using the present compounds, or pharmaceutically acceptable salts thereof, in lotions, creams, foams, patches, suspensions, solutions, and suppositories (rectal and vaginal).

G. Antilipemic Agents

Antilipemic agents, also known as antihyperlipidemic agents, which may be utilized with the methods and compositions of the invention described herein are bile acid sequestrants, fibric acid derivatives, HMG-CoA reductase inhibitors and nicotinic acid compounds. Antilipemic agents reduce the amount of cholesterol and fats in the blood through a number of mechanisms. For example, bile acid sequestrants bind to bile acids in the intestine and prevent them from being reabsorbed into the blood. The liver then produces more bile to replace the bile which has been lost. Since the body needs cholesterol to make bile, the liver uses up the cholesterol in the blood, reducing the amount of LDL cholesterol circulating in the blood.

Bile acid sequestrant agents useful with this invention include colestipol and colesevelam, and their pharmaceutically acceptable salt forms. Fibric acid derivatives which may be used with the present invention include clifofibrate, gemfibrozil and fenofibrate. HMG-CoA reductase inhibitors, also known as statins, useful with this invention include cerivastatin, fluvastatin, atorvastatin, lovastatin, pravastatin and simvastatin, or the pharmaceutically acceptable salt forms thereof. Niacin is an example of a nicotinic acid compound which may be used with the methods of this invention. Also useful are lipase inhibiting agents, such as orlistat. The use of these agents is described in further detail in U.S. Patent Pub. No. 2002/0198202-A1 (application Ser. No. 10/164,231), relevant portions thereof are herein incorporated by reference.

Bile acid sequestrant agents useful with this invention include colestipol and colesevelam, and their pharmaceutically acceptable salt forms. Colestipol is available in 1 mg COLESTID® micronized colestipol hydrochloride tablets from Pharmacia & Upjohn, with a recommended initial dose of about 2 g per day, which may be increased as need to a dose of from 2 to 16 g per day taken in divided doses. Colesevelam hydrochloride is available in 625 mg WELCHOL™ tablets from Sankyo Pharma, Inc., with a recommended starting dose of 3 tablets taken twice per day with meals or 6 tablets taken once per day with a meal. If needed, the administration may be increased to 7 tablets per day. Administration of tablets with liquid is recommended.

Fibric acid derivatives which may be used with the present invention include clifofibrate, gemfibrozil and fenofibrate. Clifofibrate is commercially available in the form of 500 mg ATROMID-S® capsules from Wyeth-Ayerst Pharmaceuticals, with a recommended daily dosage of about 2 g administered in divided doses. Gemfibrozoil is available in 600 mg LOPID® tablets from Parke-Davis, with a recommended dose for adults of about 1200 mg per day administered in two divided doses 30 minutes prior to the morning and evening meals. Fenofibrate is available in 67 mg, 134 mg and 200 mg TRICOR® tablets from Abbott Laboratories Inc., with a recommended initial dose of from 67 mg to 200 mg per day, up to a maximum daily dose of 200 mg per day.

HMG-CoA reductase inhibitors useful with this invention include cerivastatin, fluvastatin, atorvastatin, lovastatin, pravastatin and simvastatin, or the pharmaceutically acceptable salt forms thereof. BAYCOL® cerivastatin sodium tablets in 0.2 mg, 0.3 mg, 0.4 mg and 0.8 mg tablet doses are available from Bayer Corporation, with a recommended starting dose of 0.4 mg taken once daily in the evening, with a maintenance dosage range of from 0.2 mg to 0.8 mg per day. LESCOL® fluvastatin sodium capsules containing fluvastatin sodium equivalent to 20 mg or 40 mg fluvastatin are available from Novartis Pharmaceuticals Corporation with a recommended starting dose of 20 mg to 40 mg taken once daily at bedtime, and a recommended daily maintenance dose of from 20 mg to 80 mg, with a daily dose of 80 mg being taken in divided doses. LIPITOR® Atorvastatin calcium tablets are available in 10 mg, 20 mg, 40 mg or 80 mg doses from Parke Davis or Pfizer Inc., with a recommended starting dose of 10 mg taken once daily, with a final dosage range of from 10 mg to 80 mg once daily. MEVACOR® lovastatin tablets are available in 10 mg, 20 mg and 40 mg tablets from Merck & Co., Inc., with a recommended starting dose of 20 mg taken once daily with the evening meal and a recommended dosing range of from 10 mg to 80 mg per day in a single or two divided doses. PRAVACHOL® pravastatin sodium tablets are available from Bristol-Myers Squibb Company as 10 mg, 20 mg or 40 mg tablets, with a recommended starting dose of 10 mg, 20 mg or 40 mg taken once daily. ZOCOR® simvastatin tablets are available in 5 mg, 10 mg, 20 mg, 40 mg or 80 mg doses from Merck & Co., with a recommended starting dose of 20 mg per day and a maintenance dosage range of from 5 mg to 80 mg per day.

Niacin is an example of a nicotinic acid agent which may be used with the methods and compositions of this invention. It is commercially available in 500 mg, 750 mg and 1,000 mg extended release tablets under the NIASPAN® tradename from Kos Pharmaceuticals, Inc., 1001 Brickell Bay Drive, 25^{th} Floor, Miami, Fla. 33131.

Orlistat is a lipase inhibiting agent available in 120 mg capsules under the XENICAL® tradename from Roche Pharmaceuticals. Recommended dosage is one 120 mg tablet three times per day after each main meal containing fat.

H. Angiotensin Converting Enzyme (ACE) Inhibitors

ACE Inhibitors dilate blood vessels to improve the amount of blood the heart pumps and lower blood pressure. ACE inhibitors also increase blood flow, which helps to decrease the amount of work the heart has to do.

ACE inhibitors useful in the methods and compositions disclosed herein include quinapril, ramipril, verapamil, captopril, diltiazem, clonidine, hydrochlorthiazide, benazepril, prazosin, fosinopril, lisinopril, atenolol, enalapril, perindropril, perindropril tert-butylamine, trandolapril and moexipril, or a pharmaceutically acceptable salt form of one or more of these compounds. The use of these agents is described in further detail in U.S. Patent Pub. No. 2003/0055058-A1 (application Ser. No. 10/163,704), relevant portions thereof are herein incorporated by reference.

Examples include Quinapril Hydrochloride, marketed by Parke-Davis under the ACCUPRIL® tradename, which may be administered in humans at an initial dose of from about 10 to about 20 mg daily and increased over time to a range of from about 20 to 80 mg per day. Captopril tablets, containing 1-[(2S)-3-mercapto-2-methylpropionyl]-L-proline as active ingredient, may be administered at a dose of from 25 to 50 mg bid or tid. Lisinopril, available as ZESTRIL® tablets from AstraZeneca Pharmaceuticals LP, may be initiated at a dosage of about 10 mg per day and increased to a daily dose of from about 20 to 40 mg. Ramipril is available in ALTACE® capsules and may be administered at a usual maintenance dose of from about 2.5 to about 20 mg per day as a single dose or in divided doses. Verapamil HCl tablets are available in 40 mg, 80 mg and 120 mg strength under the CALAN® tradename from G.D. Searle & Co. and may be administered beginning at a dose of about 40 mg administered three times per day up to a total daily administration of about 480 mg. Dilitazem HCl capsules are available from Aventis Pharmaceuticals under the CARDIZEM® tradename.

I. Aldose Reductase Inhibitors

Aldose reductase inhibitors prevent eye and nerve damage in people with diabetes. Aldose reductase is an enzyme that is normally present in the eye and triggers the metabolism of glucose into sorbitol, which can damage the eye. Aldose reductase inhibitors slow this process.

Aldose reductase inhibitors useful in the methods and compositions of this invention include those known in the art. These include the non-limiting list of:
a) the spiro-isoquinoline-pyrrolidine tetrone compounds disclosed in U.S. Pat. No. 4,927,831 (Malamas), the contents of which are incorporated herein by reference, which includes ARI-509, also known as minalrestat or Spiro[isoquinoline-4 (1H), 3′-pyrrolidine]-1,2′,3,5′(2H)-tetrone, 2-[(4-bromo-2-fluorophenyl)methyl]-6-fluoro-(9Cl);
b) the compounds of U.S. Pat. No. 4,439,617, the contents of which are incorporated herein by reference, which includes Tolrestat, also known as Glycine, N-[[6-methoxy-5-(trifluoromethyl)-1-naphthalenyl]thioxomethyl]-N-methyl-(9Cl) or AY-27773,
c) Sorbinil (Registry No. 68367-52-2) also known as Spiro[4H-1-benzopyran-4,4′-imidazolidine]-2′,5′-dione, 6-fluoro-2,3-dihydro-, (4S)-(9Cl) or CP 45634;
d) Methosorbinil;
e) Zopolrestat, which is 1-Phthalazineacetic acid, 3,4-dihydro-4-oxo-3-[[5-(trifluoromethyl)-2-benzothiazolyl]methyl]-(9Cl) (Registry No. 110703-94-1);
f) Epalrestat, which is 3-Thiazolidineacetic acid, 5-[(2E)-2-methyl-3-phenyl-2-propenylidene]-4-oxo-2-thioxo-, (5Z)-(9Cl) (Registry No. 82159-09-9);
g) Zenarestat (Registry No. 112733-40-6) or 3-[(4-bromo-2-fluorophenyl)methyl]-7-chloro-3,4-dihydro-2,4-dioxo-1 (2H)-quinazoline acetic acid;
h) Imirestat, also known as 2,7-difluorospiro(9H-fluorene-9,4′-imidazolidine)-2′,5′-dione;
i) Ponalrestat (Registry No. 72702-95-5), which is 1-Phthalazineacetic acid, 3-[(4-bromo-2-fluorophenyl)methyl]-3,4-dihydro-4-oxo-(9Cl) and also known as Statil or Statyl;
j) ONO-2235, which is 3-Thiazolidineacetic acid, 5-[(2E)-2-methyl-3-phenyl-2-propenylidene]-4-oxo-2-thioxo-, (5Z)-(9Cl);
k) GP-1447, which is {3-[(4,5,7-trifluorobenzothiazol-2-yl)methyl]-5-methylphenylacetic acid};
l) CT-112, which is 5-(3-ethoxy-4-pentyloxyphenyl)-2,4-thiazolidinedione;
m) BAL-ARI 8, which is Glycine, N-[(7-fluoro-9-oxo-9H-xanthen-2-yl)sulfonyl]-N-methyl-(9Cl), Reg. No. 124066-40-6));
n) AD-5467, which is 2,3-dihydro-2,8-bis(1-methylethyl)-3-thioxo-4H-1,4-benzoxazine-4-acetic acid or the chloride salt form (4H-1,4-Benzoxazine-4-acetic acid, 2,3-dihydro-2,8-bis(1-methylethyl)-3-thioxo-(9Cl);
o) ZD5522, which is (3′,5′-dimethyl-4′-nitromethylsulfonyl-2-(2-tolyl)acetanilide);
p) 3,4-dihydro-2,8-diisopropyl-3-thioxo-2H-1,4-benzoxazine-4-acetic acid;
q) 1-[(3-bromo-2-benzofuranyl)sulfonyl]-2,4-imidazolidinedione (M-16209): NZ-314, which is 1-Imidazolidineacetic acid, 3-[(3-nitrophenyl)methyl]-2,4,5-trioxo-(9Cl) (Registry No. 128043-99-2);
r) 1-phthalazineacetic acid, 3,4-dihydro-4-oxo-3-[[5-trifluoromethyl)-2-benzothiazolyl]methyl]-;
s) M-79175, which is Spiro[4H-1-benzopyran-4,4′-imidazolidine]-2′,5′-dione, 6-fluoro-2,3-dihydro-2-methyl-, (2R,4S)-(9Cl) (Registry No. 102916-95-0);
t) SPR-210, which is 2H-1,4-Benzothiazine-2-acetic acid, 3,4-dihydro-3-oxo-4-[(4,5,7-trifluoro-2-benzothiazolyl)methyl]-(9Cl);
u) Spiro[pyrrolidine-3,6′(5′H)-pyrrolo[1,2,3-de][1,4]benzoxazine]-2,5,5′-trione, 8′-chloro-2′,3′-dihydro-(9Cl) (also known as ADN 138 or 8-chloro-2′,3′-dihydrospiro[pyrolizine-3,6′(5,H)-pyrolo[1,2,3-de]-[1,4]benzoxazine]2,5,5′-trione);
v) 6-fluoro-2,3-dihyro-2′,5′-dioxo-(2S-cis)-spiro[4H-1-benzopyran-4,4′-imidazolidine]-2-carboxyamide (also known as SNK-860)
   analogs, and pharmaceutically acceptable salt forms of one or more of these compounds. The use of these agents is described in further detail in U.S. Patent Pub. No. 2002/0198201-A1 (application Ser. No. 10/164,214), relevant portions thereof are herein incorporated by reference.

Among the aldose reductase inhibitors of this invention are minalrestat Tolrestat, Sorbinil, Methosorbinil, Zopolrestat, Epalrestat, Zenarestat Imirestat, and Ponalrestat or the pharmaceutically acceptable salt forms thereof.

Aldose reductase inhibitors useful with this invention may be administered by the dosages and regimens known in the art. For instance, minalrestat (ARI-509) may be administered in oral dosages of from about 0.1 mg/kg of body weight to about 1.0 mg/kg of body weight per day. Tolrestat has been administered in human patients at a single daily oral dose of 200 mg (Troy et al., Clin. Pharmacol. Ther. 51:271-277 (1992) or 200 mg/twice a day (van Griensven et al., Clin. Pharmacol. Ther. 58:631-640 (1995)). Sorbinil has been administered in humans at 50 mg and 200 mg daily doses (Christensen et al., Acta Neurologica Scandinavica 71:164-167 (1985)). Zopolrestat has been administered in humans at doses ranging from 50 mg to 1200 mg per day (Inskeep et al., J. Clin. Pharmacol. 34:760-766 (1994)). Zenalrestat has been administered to human patients in doses of 150 mg, 300 mg and 600 mg, each given twice daily (Greene et al., Neurology 53:580-591 (1999)). Imirestat has been administered to humans at doses from 2 mg to 50 mg per day (Brazzell et al., Pharm. Res. 8:112-118 (1991)). Ponalrestat has been administered to humans at a daily dose of 600 mg (Airey et al., Diabetic Medicine 6:804-808 (1989)).

IV. Combination Therapy

A. Treatment of Obesity, Cardiovascular Diseases, or Disorders of Insulin Metabolism

In combination therapy methods described herein, at least one GDF-8 inhibitor is administered with at least one other therapeutic agent as provided above. The combination therapy may also include a combination of more than one GDF-8 inhibitor and/or more than one therapeutic agents.

The combination therapy can be administered simultaneously or sequentially. Simultaneous administration requires the administration of at least one dose of each of the GDF-8 inhibitor and at least one therapeutic agent at the same time or times. Sequential administration may include a bolus dosage of the GDF-8 inhibitor followed by multiple doses of at least one therapeutic agent over time; it may also include multiple doses of both compounds. Varying the dosage pattern may vary the results in achieving the desired treatment goal.

B. Evaluation of Combination Therapy

The data obtained from cell culture assays and animal studies can be used in formulating a range of dosage for use in humans. The dosage of such compounds may lie within a range of circulating concentrations that include the ED₅₀ with little or no toxicity. The dosage may vary within this range depending upon the dosage form employed and the route of administration utilized. For any compound used in the present invention, the therapeutically effective dose can be estimated initially from cell culture assays. A dose may be formulated in animal models to achieve a circulating plasma concentration range that includes the IC₅₀ (i.e., the concentration of the test compound or compounds which achieves a half-maximal inhibition of symptoms) as determined in cell culture. Levels in plasma may be measured, for example, by high performance liquid chromatography. The effects of any particular dosage can be monitored by a suitable bioassay. Examples of suitable bioassays include DNA replication assays, transcription-based assays, GDF-8 protein/receptor binding assays, creatine kinase assays, assays based on the differentiation of pre-adipocytes, assays based on glucose uptake in adipocytes, and immunological assays.

Prior to administration to patients, a given combination therapy can be evaluated in a therapeutic animal model, such as in the obese Zucker diabetic rats described in Park, Circulation 104:815-819 (2001). The obese Zucker rat is characterized by excessive body weight, insulin resistance, hyperinsulinemia, and mild hyperglycemia, and is a well-established model of type 2 diabetes. Obese Zucker rats aged 8 to 9 weeks are used as the diabetic model, and lean Zucker rats aged 11 to 14 weeks are used as controls. The combination therapy can be administered to the rats following the treatment plan sought to be evaluated. Investigators can then track blood chemistry and morphology changes over time to assess effectiveness. (Park, at 818).

In any given patient, or as part of a clinical study, the effectiveness of combination therapy can be measured using parameter including plasma LDL cholesterol level, total cholesterol level, triglyceride level, insulin uptake, blood pressure, and blood glucose levels. Such tests are easily undertaken as part of the clinical regimen of evaluating and following-up with any patient. Dosages of each therapeutic in the combination therapy can be adjusted in accord with the evaluation.

### EXAMPLES

### Example 1

### A Combination Therapy to Treat Diabetes

A patient with diabetes is treated with a combination of an antibody against GDF-8, such as Myo-29, administered in a 1 mg/kg bolus weekly for 4 weeks and metphormin, administered 500 mg, twice a day.

### Example 2

### A Combination Therapy to Treat Obesity

A patient with obesity is treated with a combination of an antibody against GDF-8, such as JA-16, administered in a 1 mg/kg bolus weekly for 4 weeks and Lipitor, administered 10 mg a day.

### Example 3

### A Combination Therapy to Treat Diabetes

A patient with diabetes is treated with a modified soluble receptor fusion, such as an ActRIIB-Fc fusion, administered 100 μg/kg weekly for 4 weeks and pioglitazone, administered 50 mg, twice a day.

### Example 4

### A Combination Therapy to Treat Cardiovascular Disease

A patient with cardiovascular disease secondary to type 2 diabetes is treated with a combination of LOPID, 600 mg twice per day and GDF-8 propeptide Fc fusion inhibitor, administered in a 5 mg/kg bolus weekly for 4 weeks.

### Example 5

### A Combination Therapy to Treat Type 2 Diabetes

A patient with type 2 diabetes is treated with a combination of a mutated GDF-8 propeptide, such as the propeptide with a mutation in at least one amino acid whereby the propeptide's proteolytic cleavage at an aspartate residue corresponding to Asp-19 in SEQ ID NO:65 is reduced relative to that of a corresponding unmodified GDF-8 propeptide, administered in a 10 mg/kg bolus weekly for 4 weeks, AMARYL, 1 mg per day and insulin, taken as needed.

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications and patents cited in this disclosure are incorporated by reference in their entirety. To the extent the material incorporated by reference contradicts or is inconsistent with this specification, the specification will supercede any such material. The citation of any references herein is not an admission that such references are prior art to the present invention.

Unless otherwise indicated, all numbers expressing quantities of ingredients, reaction conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

We claim:

1. A method of treating a targeted syndrome in a subject, comprising administering to the subject a therapeutically effective amount of at least one GDF-8 inhibitor, and a therapeutically effective amount of at least one other therapeutic agent which treats the targeted syndrome.

2. A method according to claim 1, wherein the targeted syndrome is chosen from at least one of obesity, cardiovascular diseases, and disorders of insulin metabolism.

3. A method according to claim 1, wherein the GDF-8 inhibitor is chosen from at least one of an antibody against GDF-8, an antibody against GDF-8 receptor, a modified soluble receptor, a protein binding to GDF-8, a protein binding to GDF-8 receptor, inhibitors of protease activation of the GDF-8 small latent complex, and GDF-8 inhibiting mimetics thereof.

4. A method according to claim 3, wherein the GDF-8 inhibitor specifically binds a mature GDF-8 protein.

5. The method according to claim 1, wherein the therapeutic agent is chosen from at least one of an angiotensin converting enzyme (ACE) inhibitor, a sulfonylurea agent, an antilipemic agent, a biguanide agent, a thiazolidinedione agent, insulin, an alpha-glucosidase inhibitor, an aldose reductase inhibitor, or a PTPase inhibitor.

6. The method of claim 5, wherein the angiotensin converting enzyme (ACE) inhibitor is chosen from at least one of quinapril, ramipril, verapamil, captopril, diltiazem, clonidine, hydrochlorthiazide, benazepril, prazosin, fosinopril, lisinopril, atenolol, enalapril, perindropril, perindropril tert-butylamine, trandolapril and moexipril, and the suitable pharmaceutically acceptable salt forms thereof.

7. The method of claim 5, wherein the sulfonylurea agent is chosen from at least one of glipizide, glyburide (glibenclamide), chlorpropamide, tolbutamide, tolazamide and glimepriride, and the pharmaceutically acceptable salt forms thereof.

8. The method of claim 5, wherein the antilipemic agent is chosen from at least one of bile acid sequestrants, fibric acid derivatives, HMG-CoA reductase inhibitors and nicotinic acid compounds, and the pharmaceutically acceptable salt forms thereof.

9. The method of claim 5, wherein the biguanide agent is chosen from at least one of metformin and its pharmaceutically acceptable salt forms.

10. The method of claim 5, wherein the thiazolidinedione agent is chosen from at least one of pioglitazone and rosiglitazone, and the pharmaceutically acceptable salt forms thereof.

11. The method of claim 5, wherein the insulin is chosen from at least one of rapid acting insulins, intermediate acting insulins, long acting insulins and combinations of intermediate and rapid acting insulins.

12. The method of claim 5, wherein the alpha-glucosidase inhibitor is chosen from at least one of miglitol and acarbose, and the pharmaceutically acceptable salt forms thereof.

13. The method of claim 5, wherein the aldose reductase inhibitor is chosen from at least one of:
a) a spiro-isoquinoline-pyrrolidine tetrone compound;
b) 2-[(4-bromo-2-fluorophenyl)methyl]-6-fluoro-(9Cl);
c) Tolrestat,
d) Sorbinil;
e) Methosorbinil;
f) Zopolrestat;
g) Epalrestat;
h) Zenarestat;
i) Imirestat;
j) Ponalrestat;
k) ONO-2235;
l) GP-1447;
m) CT-112;
n) BAL-ARI 8;
o) AD-5467;
p) ZD5522;
q) 3,4-dihydro-2,8-diisopropyl-3-thioxo-2H-1,4-benzoxazine-4-acetic acid;
r) 1-[(3-bromo-2-benzofuranyl)sulfonyl]-2,4-imidazolidinedione (M-16209): NZ-314, which is 1-Imidazolidineacetic acid, 3-[(3-nitrophenyl)methyl]-2,4,5-trioxo-(9Cl);
s) 1-phthalazineacetic acid, 3,4-dihydro-4-oxo-3-[[5-trifluoromethyl)-2-benzothiazolyl]methyl]-;
t) M-79175;
u) SPR-210;
v) Spiro[pyrrolidine-3,6′(5′H)-pyrrolo[1,2,3-de][1,4]benzoxazine]-2,5,5′-trione, 8′-chloro-2′,3′-dihydro-(9Cl);
w) 6-fluoro-2,3-dihyro-2′,5′-dioxo-(2S-cis)-spiro[4H-I-benzopyran-4,4′-imidazolidine]-2-carboxyamide; and
x) analogs and pharmaceutically acceptable salts thereof.

14. The method of claim 5, wherein the PTPase inhibitor is chosen from at least one compound with the formula (I): 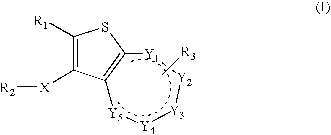
R₁ is C(O)OR₇, 5- to 6-membered heterocycle, H, halogen, CN, or C(O)NR₇R₈;
R₂ is C(O)ZR₄ or CN;
Z is —O— or —NR₅—;
X is —O—C₁₋₃alkylene-, —NR₈—C₁₋₃alkylene-, —S—C₁₋₃alkylene-, —SO—C₁₋₃alkylene-, —SO₂—C₁₋₃alkylene-, —C₁₋₄alkylene-, —C₂₋₄alkenylene-, or —C₂₋₄alkynylene-, wherein any of the alkylene, alkenylene and alkynylene groups can be optionally substituted with one or more halogen, oxo, HN═, CN, OCF₃, OH, NH₂, NO₂, R₄, or Q;
each Y₁, Y₂, Y₃, Y₄, and Y₅ is, independently, CR₃, N, S, or O, one or two of Y₁, Y₂, Y₃, Y₄, and Y₅ can be absent;
each R₃ is, independently, H, aryl, 5- to 8-membered heterocyclyl, C₁₋₆alkyl, C₂₋₆alkenyl, C₂₋₆alkynyl, halogen, CN, OCF₃, OH, NH₂, NO₂, or Q, wherein any of the aryl, heterocyclic, alkyl, alkenyl or alkynyl groups is optionally substituted with one or more halogen, oxo, CN, OCF₃, OH, NH₂, NO₂, N₃, R₄, or Q;
each Q is, independently, —OC(O)NR₄R₅, —OR₄, —OC(O)R₄, —COOR₄, —C(O)NR₄R₅, —C(O)R₄, —C(═N—OH)R₄, —NR₄R₅, —N⁺R₄R₅R₆, —NR₄C(O)R₅, —NR₄C(O)NR₅R₆, —NR₄C(O)OR₅, —NR₄S(O)₂R₅, —SR₄, —S(O)R₄, —S(O)₂R₄, or —S(O)₂NR₄R₅;
each R₄, R₅, and R₆ is, independently, H, C₁₋₁₆alkyl, C₂₋₁₂alkenyl, C₂₋₁₂alkynyl, C₃₋₈cycloalkyl, cycloalkylC₁₋₆alkyl, 5- to 8-membered heterocycle, heterocyclicC₁₋₆alkyl, aryl, arylC₁₋₆alkyl, arylC₂₋₆alkenyl, or arylC₂₋₆alkynyl, each R₄, R₅, and R₆ can be optionally substituted with one or more C₁₋₆alkyl, C₂₋₆alkenyl, C₂₋₆alkynyl, halogen, oxo, CN, OCF₃, OH, NH₂, NO₂, N₃, —OC(O)NR₇R₈, —OR₇, —OC(O)R₇, —COOR₇, —C(O)NR₇R₈, —C(O)R₇, —NR₇R₈, —N⁺R₇R₈R₉, —NR₇C(O)R₈, —NR₇C(O)NR₈R₉, —NR₇C(O)OR₈, —NR₇S(O)₂R₈, —SR₇, —S(O)R₇, —S(O)₂R₇, or —S(O)₂NR₇R₈;
each R₇, R₈, and R₉ is, independently, H, C₁₋₁₂alkyl, C₂₋₁₂alkenyl, C₂₋₁₂alkynyl, C₃₋₁₂cycloalkyl, aryl, or arylC₁₋₁₂alkyl, each R₇, R₈, and R₉ can be optionally substituted with one or more halogen, oxo, CN, OCF₃, OH, NH₂, or NO₂;
when the ring system is 1-benzothiophene, R₁ is C(O)OCH₃, and X is —OCH₂—, then R₂ is not C(O)OCH₃;
when the ring system is 1-benzothiophene, R₁ is C(O)OH, and X is —OCH₂—, then R₂ is not C(O)OH;
when the ring system is thieno[2,3-b]pyridine, R₁ is isopropyl ester, and X is —OCH₂—, then R₂ is not C₁₋₃alkyl ester;
when the ring system is thieno[2,3-b]pyridine, R, is C(O)OC₁₋₄alkyl, and X is —OCH₂— or —OCH(CH₃)—, then R₂ is not CN;
when the ring system is thieno[2,3-b]pyridine, R, is isopropyl ester, and X is —SCH₂CH₂—, then R₂ is not CN; and
when the ring system is thieno[2,3-b]pyridine, R₁ is isopropyl ester, and X is —SCH₂—, then R₂ is not isopropyl ester.

15. The method of claim 5, wherein the PTPase inhibitor is chosen from at least one compound with the formula (II): 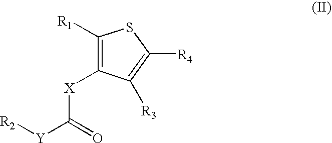
R₁ is R₅, OR₅, C(O)OR₅, C(O)R₅, or C(O)NR₅R₆;
R₂ is R₅;
X is —O—C₁₋₃alkylene-, —NR₈—C₁₋₃alkylene-, —S—C₁₋₃alkylene-, —SO—C₁₋₃alkylene-, —SO₂—C₁₋₃alkylene-, —C₁₋₄alkylene-, —C₂₋₄alkenylene-, or —C₂₋₄alkynylene-, wherein any of the alkylene, alkenylene or alkynylene groups can be optionally substituted with one or more halogen, oxo, imido, CN, OCF₃, OH, NH₂, NO₂, or Q;
Y is absent, —O—, or —NR₆—;
R₃ is H, halogen, CN, CF₃, OCF₃, C₁₋₃ alkyl, C₃₋₄cycloalkyl, C₁₋₃alkoxy, or aryl;
R₄ is A-B-E-D, where A is absent or arylene, heteroarylene, C₁₋₆alkylene, C₂₋₆ alkenyldiyl, or C₂₋₆alkynyl, each A can be optionally substituted with one or more of C₁₋₆alkyl, C₂₋₆alkenyl, C₂₋₆alkynyl, halogen, CN, OCF₃, OH, NH₂, CHO, NO₂, or Q, any of the alkyl, alkenyl or alkynyl groups is optionally substituted with one or more halogen, oxo, CN, OCF₃, OH, NH₂, NO₂, N₃, or Q;
each A can be optionally terminated with one or more arylene, alkylene, or alkenylene;
B is absent or —NR₅—, —NR₇—, —N(R₅)CH₂—, —N(R₇)CH₂—, —N(R₉)—, —N(R₉)C(O)—, —N(R₉)C(O)C(R₁₁)(R₁₂)—, —N(R₉)C(O)C(O)—, —N(R₉)C(O)N(R₁₀)—, —N(R₉)SO₂—, —N(R₉)SO₂C(R₁₀)(R₁₁)—, —N(R₉)(R₁₀)C(R₁₁)(R₁₂)—, —N(R₉)C(R₁₁)(R₁₂)C(R₁₃)(R₁₄)—, —O—, —O—C(R₁₁)(R₁₂)—, —O—C(R₁₁)(R₁₂)C(R₁₃)(R₁₄)—, —C(R₁₁)(R₁₂)—O—, —C(R₁₁)(R₁₂)—O—C(R₁₃)(R₁₄)—, —C(R₁₁)(R₁₂)N(R₉)—, —C(R₁₁)(R₁₂)N(R₉)C(R₁₃)(R₁₄)—, —C(R₁₁)(R₁₂)S—, —C(R₁₁)(R₁₂)SC(R₁₃)(R₁₄)—, or —C(R₁₁)(R₁₂)SO₂C(R₁₃)(R₁₄)—;
E is absent or C₃₋₁₂cycloalkylene, 3- to 12-membered heterocycdiyl, arylene, C₁₋₁₂alkylene, C₂₋₁₂alkenylene, or C₂₋₁₂alkynylene, where each E is optionally substituted with one or more C₁₋₃alkyl, C₁₋₃alkoxy, halogen, CN, OH, NH₂, or NO₂;
D is one or more H, halogen, OH, NH₂, CHO, CN, NO₂, CF₃, or Q;
when A, B, and E are absent, R₁ is C(O)OH or C(O)OCH₃, R₂ is H, and R₃ is H or chlorine, D is not H or chlorine; and when A, B, and E are absent, R₁ is C(O)OH or C(O)OCH₃, R₂ is H, and R₃ is H or bromine, D is not H or bromine;
each Q, independently, is —R₅, —R₇, —OR₅, —OR₇, —NR₅R₆, —NR₅R₇, —N⁺R₅R₆R₈, S(O)ₙR₅, or —S(O)ₙR₇, and n is 0, 1, or 2;
each R₅, R₆, and R₈, independently, is H, C₁₋₁₂alkyl, C₂₋₁₂alkenyl, C₂₋₁₂alkynyl, C₃₋₁₂cycloalkyl, C₁₋₁₂alkoxyC₁₋₁₂alkyl, cycloalkylC₁₋₆alkyl, 3- to 8-membered heterocycyl, heterocycylC₁₋₆alkyl, aryl, arylC₁₋₆ alkyl, arylC₂₋₆ alkenyl, or arylC₂₋₆ alkynyl, each R₅, R₆, and R₈ can be optionally substituted with one or more R₉, —OR₉, —OC(O)OR₉, —C(O)R₉, —C(O)OR₉, —C(O)NR₉R₁₀, —SR₉, —S(O)R₉, —S(O)₂R₉, —NR₉R₁₀, —N⁺R₉R₁₀R₁₁, —NR₉C(O)R₁₀, —NC(O)NR₉R₁₀, —NR₉S(O)₂R₁₀, oxo, halogen, CN, OCF₃, CF₃, OH, or NO₂;
R₇ is —C(O)R₅, —C(O)OR₅, —C(O)NR₅R₆, —S(O)₂R₅, —S(O)R₅, or —S(O)₂NR₅R₆;
each R₉, R₁₀, R₁₁, R₁₂, R₁₃ and R₁₄ is, independently, H, C₁₋₁₂alkyl, C₂₋₁₂alkenyl, C₂₋₁₂alkynyl, C₃₋₁₂cycloalkyl, aryl, or arylC₁₋₁₂alkyl, any of the alkyl, alkenyl, alkynyl, cycloalkyl, aryl, or arylalkyl groups is optionally substituted with one or more halogen, oxo, CN, OCF₃, OH, NH₂, or NO₂.

16. The method of claim 1, wherein the administration is sequential.

17. The method of claim 1, wherein the administration is simultaneous.

18. The method of claim 1, wherein the administration of at least one therapeutic agent is oral.

19. The method of claim 1, wherein the administration is parenteral.

20. The method of claim 19, wherein the parenteral administration is intravenous.

21. A pharmaceutical composition useful for treating a targeted syndrome comprising combining a therapeutically effective amount of a GDF-8 inhibitor and a therapeutically effective amount of at least one other therapeutic agent which treats the targeted syndrome.

22. A pharmaceutical composition according to claim 21, wherein the GDF-8 inhibitor is chosen from at least one of an antibody against GDF-8, an antibody against GDF-8 receptor, a modified soluble receptor, a protein binding to GDF-8, a protein binding to GDF-8 receptor, inhibitors of protease activation of the GDF-8 small latent complex, and GDF-8 inhibiting mimetics thereof.

23. A pharmaceutical composition according to claim 22 wherein the protein binding to GDF-8 is chosen from at least one of a GDF-8 propeptide having SEQ ID NO:65, a mutated GDF-8 propeptide, follistatin, follistatin-domain containing proteins, and Fc fusions thereof.

24. A pharmaceutical composition according to claim 22, wherein the GDF-8 inhibitor specifically binds a mature GDF-8 protein.

25. The pharmaceutical composition according to claim 21, wherein the therapeutic agent is chosen from at least one of an angiotensin converting enzyme (ACE) inhibitor, a sulfonylurea agent, an antilipemic agent, a biguanide agent, a thiazolidinedione agent, insulin, an alpha-glucosidase inhibitor, an aldose reductase inhibitor, or a PTPase inhibitor.

26. The pharmaceutical composition of claim 25, wherein the angiotensin converting enzyme (ACE) inhibitor is chosen from at least one of quinapril, ramipril, verapamil, captopril, diltiazem, clonidine, hydrochlorthiazide, benazepril, prazosin, fosinopril, lisinopril, atenolol, enalapril, perindropril, perindropril tert-butylamine, trandolapril and moexipril, or a pharmaceutically acceptable salt form of one or more of these compounds.

27. The pharmaceutical composition of claim 25, wherein the sulfonylurea agent is chosen from at least one of glipizide, glyburide (glibenclamide), chlorpropamide, tolbutamide, tolazamide and glimepriride, and the pharmaceutically acceptable salt forms thereof.

28. The pharmaceutical composition of claim 25, wherein the antilipemic agent is chosen from at least one of bile acid sequestrants, fibric acid derivatives, HMG-CoA reductase inhibitors and nicotinic acid compounds, and the pharmaceutically acceptable salt forms thereof.

29. The pharmaceutical composition of claim 25, wherein the biguanide agent is chosen from at least one of metformin and its pharmaceutically acceptable salt forms.

30. The pharmaceutical composition of claim 25, wherein the thiazolidinedione agent is chosen from at least one of pioglitazone and rosiglitazone, and pharmaceutically acceptable salt forms of these agents.

31. The pharmaceutical composition of claim 25, wherein the insulin is chosen from at least one of rapid acting insulins, intermediate acting insulins, long acting insulins and combinations of intermediate and rapid acting insulins.

32. The pharmaceutical composition of claim 25, wherein the alpha-glucosidase inhibitor is chosen from at least one of miglitol and acarbose, and a pharmaceutically acceptable salt form of one or more of these compounds.

33. The pharmaceutical composition of claim 25, wherein the aldose reductase inhibitor is chosen from at least one of
a) a spiro-isoquinoline-pyrrolidine tetrone compound;
b) 2-[(4-bromo-2-fluorophenyl)methyl]-6-fluoro-(9Cl);
c) Tolrestat,
d) Sorbinil;
e) Methosorbinil;
f) Zopolrestat;
g) Epalrestat;
h) Zenarestat;
i) Imirestat;
j) Ponalrestat;
k) ONO-2235;
l) GP-1447;
m) CT-112;
n) BAL-ARI 8;
o) AD-5467;
p) ZD5522;
q) 3,4-dihydro-2,8-diisopropyl-3-thioxo-2H-1,4-benzoxazine-4-acetic acid;
r) 1-[(3-bromo-2-benzofuranyl)sulfonyl]-2,4-imidazolidinedione (M-16209): NZ-314, which is 1-Imidazolidineacetic acid, 3-[(3-nitrophenyl)methyl]-2,4,5-trioxo-(9Cl);
s) 1-phthalazineacetic acid, 3,4-dihydro-4-oxo-3-[[5-trifluoromethyl)-2-benzothiazolyl]methyl]-;
t) M-79175;
u) SPR-210;
v) Spiro[pyrrolidine-3,6′(5′H)-pyrrolo[1,2,3-de][1,4]benzoxazine]-2,5,5′-trione, 8′-chloro-2′,3′-dihydro-(9Cl);
w) 6-fluoro-2,3-dihyro-2′,5′-dioxo-(2S-cis)-spiro[4H-1-benzopyran-4,4′-imidazolidine]-2-carboxyamide;
analogs and pharmaceutically acceptable salts thereof.

34. The pharmaceutical composition of claim 25, wherein the PTPase inhibitor is chosen from at least one compound with the formula (I): 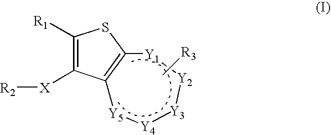
R₁ is C(O)OR₇, 5- to 6-membered heterocycle, H, halogen, CN, or C(O)NR₇R₈;
R₂ is C(O)ZR₄ or CN;
Z is —O— or —NR₅—;
X is —O—C₁₋₃alkylene-, —NR₈—C₁₋₃alkylene-, —S—C₁₋₃alkylene-, —SO—C₁₋₃alkylene-, —SO₂—C₁₋₃alkylene-, —C₁₋₄alkylene-, —C₂₋₄alkenylene-, or —C₂₋₄alkynylene-, wherein any of the alkylene, alkenylene and alkynylene groups can be optionally substituted with one or more halogen, oxo, HN═, CN, OCF₃, OH, NH₂, NO₂, R₄, or Q;
each Y₁, Y₂, Y₃, Y₄, and Y₅ is, independently, CR₃, N, S, or O, one or two of Y₁, Y₂, Y₃, Y₄, and Y₅ can be absent;
each R₃ is, independently, H, aryl, 5- to 8-membered heterocyclyl, C₁₋₆alkyl, C₂₋₆alkenyl, C₂₋₆alkynyl, halogen, CN, OCF₃, OH, NH₂, NO₂, or Q, wherein any of the aryl, heterocyclic, alkyl, alkenyl or alkynyl groups is optionally substituted with one or more halogen, oxo, CN, OCF₃, OH, NH₂, NO₂, N₃, R₄, or Q;
each Q is, independently, —OC(O)NR₄R₅, —OR₄, —OC(O)R₄, —COOR₄, —C(O)NR₄R₅, —C(O)R₄, —C(═N—OH)R₄, —NR₄R₅, —N⁺R₄R₅R₆, —NR₄C(O)R₅, —NR₄C(O)NR₅R₆, —NR₄C(O)OR₅, —NR₄S(O)₂R₅, —SR₄, —S(O)R₄, —S(O)₂R₄, or —S(O)₂NR₄R₅;
each R₄, R₅, and R₆ is, independently, H, C₁₋₁₆alkyl, C₂₋₁₂alkenyl, C₂₋₁₂alkynyl, C₃₋₈cycloalkyl, cycloalkylC₁₋₆alkyl, 5- to 8-membered heterocycle, heterocyclicC₁₋₆alkyl, aryl, arylC₁₋₆alkyl, arylC₂₋₆alkenyl, or arylC₂₋₆alkynyl, each R₄, R₅, and R₆ can be optionally substituted with one or more C₁₋₆alkyl, C₂₋₆alkenyl, C₂₋₆alkynyl, halogen, oxo, CN, OCF₃, OH, NH₂, NO₂, N₃, —OC(O)NR₇R₈, —OR₇, —OC(O)R₇, —COOR₇, —C(O)NR₇R₈, —C(O)R₇, —NR₇R₈, —N⁺R₇R₈R₉, —NR₇C(O)R₈, —NR₇C(O)NR₈R₉, —NR₇C(O)OR₈, —NR₇S(O)₂R₈, —SR₇, —S(O)R₇, —S(O)₂R₇, or —S(O)₂NR₇R₈;
each R₇, R₈, and R₉ is, independently, H, C₁₋₁₂alkyl, C₂₋₁₂alkenyl, C₂₋₁₂alkynyl, C₃₋₁₂cycloalkyl, aryl, or arylC₁₋₁₂alkyl, each R₇, R₈, and R₉ can be optionally substituted with one or more halogen, oxo, CN, OCF₃, OH, NH₂, or NO₂;
when the ring system is 1-benzothiophene, R₁ is C(O)OCH₃, and X is —OCH₂—, then R₂ is not C(O)OCH₃;
when the ring system is 1-benzothiophene, R₁ is C(O)OH, and X is —OCH₂—, then R₂ is not C(O)OH;
when the ring system is thieno[2,3-b]pyridine, R₁ is isopropyl ester, and X is —OCH₂—, then R₂ is not C₁₋₃alkyl ester;
when the ring system is thieno[2,3-b]pyridine, R₁ is C(O)OC₁₋₄alkyl, and X is —OCH₂— or —OCH(CH₃)—, then R₂ is not CN;
when the ring system is thieno[2,3-b]pyridine, R₁ is isopropyl ester, and X is —SCH₂CH₂—, then R₂ is not CN; and
when the ring system is thieno[2,3-b]pyridine, R₁ is isopropyl ester, and X is —SCH₂—, then R₂ is not isopropyl ester.

35. The pharmaceutical composition of claim 25, wherein the PTPase inhibitor is chosen from at least one compound with the formula (II): 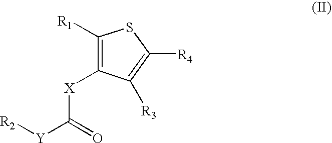
R₁ is R₅, OR₅, C(O)OR₅, C(O)R₅, or C(O)NR₅R₆;
R₂ is R₅;
X is —O—C₁₋₃alkylene-, —NR₈—C₁₋₃alkylene-, —S—C₁₋₃alkylene-, —SO—C₁₋₃alkylene-, —SO₂—C₁₋₃alkylene-, —C₁₋₄alkylene-, —C₂₋₄alkenylene-, or —C₂₋₄alkynylene-, wherein any of the alkylene, alkenylene or alkynylene groups can be optionally substituted with one or more halogen, oxo, imido, CN, OCF₃, OH, NH₂, NO₂, or Q;
Y is absent, —O—, or —NR₆—;
R₃ is H, halogen, CN, CF₃, OCF₃, C₁₋₃ alkyl, C₃₋₄cycloalkyl, C₁₋₃alkoxy, or aryl;
R₄ is A-B-E-D, where A is absent or arylene, heteroarylene, C₁₋₆alkylene, C₂₋₆ alkenyldiyl, or C₂₋₆alkynyl, each A can be optionally substituted with one or more of C₁₋₆alkyl, C₂₋₆alkenyl, C₂₋₆alkynyl, halogen, CN, OCF₃, OH, NH₂, CHO, NO₂, or Q, any of the alkyl, alkenyl or alkynyl groups is optionally substituted with one or more halogen, oxo, CN, OCF₃, OH, NH₂, NO₂, N₃, or Q;
each A can be optionally terminated with one or more arylene, alkylene, or alkenylene;
B is absent or —NR₅—, —NR₇—, —N(R₅)CH₂—, —N(R₇)CH₂—, —N(R₉)—, —N(R₉)C(O)—, —N(R₉)C(O)C(R₁₁)(R₁₂)—, —N(R₉)C(O)C(O)—, —N(R₉)C(O)N(R₁₀)—, —N(R₉)SO₂—, —N(R₉)SO₂C(R₁₀)(R₁₁)—, —N(R₉)(R₁₀)C(R₁₁)(R₁₂)—, —N(R₉)C(R₁₁)(R₁₂)C(R₁₃)(R₁₄)—, —O—, —O—C(R₁₁)(R₁₂)—, —O—C(R₁₁)(R₁₂)C(R₁₃)(R₁₄)—, —C(R₁₁)(R₁₂)—O—, —C(R₁₁)(R₁₂)—O—C(R₁₃)(R₁₄)—, —C(R₁₁)(R₁₂)N(R₉)—, —C(R₁₁)(R₁₂)N(R₉)C(R₁₃)(R₁₄)—, —C(R₁₁)(R₁₂)S—, —C(R₁₁)(R₁₂)SC(R₁₃)(R₁₄)—, or —C(R₁₁)(R₁₂)SO₂C(R₁₃)(R₁₄)—;
E is absent or C₃₋₁₂cycloalkylene, 3- to 12-membered heterocycdiyl, arylene, C₁₋₁₂alkylene, C₂₋₁₂alkenylene, or C₂₋₁₂alkynylene, where each E is optionally substituted with one or more C₁₋₃alkyl, C₁₋₃alkoxy, halogen, CN, OH, NH₂, or NO₂;
D is one or more H, halogen, OH, NH₂, CHO, CN, NO₂, CF₃, or Q;
when A, B, and E are absent, R₁ is C(O)OH or C(O)OCH₃, R₂ is H, and R₃ is H or chlorine, D is not H or chlorine; and when A, B, and E are absent, R₁ is C(O)OH or C(O)OCH₃, R₂ is H, and R₃ is H or bromine, D is not H or bromine;
each Q, independently, is —R₅, —R₇, —OR₅, —OR₇, —NR₅R₆, —NR₅R₇, —N⁺R₅R₆R₈, S(O)ₙR₅, or —S(O)ₙR₇, and n is 0, 1, or 2;
each R₅, R₆, and R₈, independently, is H, C₁₋₁₂alkyl, C₂₋₁₂alkenyl, C₂₋₁₂alkynyl, C₃₋₁₂cycloalkyl, C₁₋₁₂alkoxyC₁₋₁₂alkyl, cycloalkylC₁₋₆alkyl, 3- to 8-membered heterocycyl, heterocycylC₁₋₆alkyl, aryl, arylC₁₋₆ alkyl, arylC₂₋₆ alkenyl, or arylC₂₋₆ alkynyl, each R₅, R₆, and R₈ can be optionally substituted with one or more R₉, —OR₉, —OC(O)OR₉, —C(O)R₉, —C(O)OR₉, —C(O)NR₉R₁₀, —SR₉, —S(O)R₉, —S(O)₂R₉, —NR₉R₁₀, —N⁺R₉R₁₀R₁₁, —NR₉C(O)R₁₀, —NC(O)NR₉R₁₀, —NR₉S(O)₂R₁₀, oxo, halogen, CN, OCF₃, CF₃, OH, or NO₂;
R₇ is —C(O)R₅, —C(O)OR₅, —C(O)NR₅R₆, —S(O)₂R₅, —S(O)R₅, or —S(O)₂NR₅R₆;
each R₉, R₁₀, R₁₁, R₁₂, R₁₃ and R₁₄ is, independently, H, C₁₋₁₂alkyl, C₂₋₁₂alkenyl, C₂₋₁₂alkynyl, C₃₋₁₂cycloalkyl, aryl, or arylC₁₋₁₂alkyl, any of the alkyl, alkenyl, alkynyl, cycloalkyl, aryl, or arylalkyl groups is optionally substituted with one or more halogen, oxo, CN, OCF₃, OH, NH₂, or NO₂.
